(19) United States
(12) Patent Application Publication
(10) Pub. No.: US 2026/0256858 A1
(43) Pub. Date: Sep. 3, 2026

(54) Amanita Muscaria Mushroom Extracts and Products, and Improved Processes for Producing Extracts of Amanita Muscaria Mushrooms at Kilogram Scale

(71) Applicant: Psyched Wellness Ltd., Toronto (CA)

(72) Inventors: Jeffrey STEVENS, Toronto (CA); Brian TANCOWNY, Beaumont (CA); Deepank UTKHEDE, Surrey (CA)

(21) Appl. No.: 19/157,676

(22) PCT Filed: Feb. 16, 2024
(86) PCT No.: PCT/US2024/016316

§ 371 Date: Aug. 18, 2025

Related U.S. Application Data
(60) Provisional application No. 63/446,343, filed on Feb. 16, 2023.

(51) Int. Cl. A61K 36/07; A23G 3/36; A23L 2/56; A61K 9/00; A61K 9/06; A61K 31/198; A61K 31/42; A61K 33/06; B01D 69/02; B01D 69/04; C12Q 1/06; G01N 30/72; G01N 33/68; G01N 33/84; H01J 49/10

(52) U.S. Cl. CPC A61K 36/07; A23G 3/368; A23L 2/56; A61K 9/0014; A61K 9/0056; A61K 9/0078; A61K 9/0095; A61K 9/06; A61K 31/198; A61K 31/42; A61K 33/06; B01D 69/02; B01D 69/04; C12Q 1/06; G01N 33/6812; G01N 33/84; A61K 2236/15; A61K 2236/17; A61K 2236/331; A61K 2236/37; A61K 2236/53; B01D 2325/0283; G01N 30/72; G01N 2333/245; G01N 2333/255; G01N 2333/375; H01J 49/105

(57) ABSTRACT

Disclosed herein are processes for extracting muscimol from Amanita mushrooms, and more particularly, from Amanita muscaria, including processes that increase muscimol content and decrease ibotenic acid content, and further including improved processes that produce extract at scale, such as at kilogram scale. In some aspects are methods for selecting Amanita mushroom biomass suitable for the disclosed extraction methods, so as to produce extracts and products that are safe and beneficial for human use. Also disclosed are numerous compositions and formulations made with disclosed extracts, including beverage formulations, having a variety of improvements and advantages over previously known products made from Amanita muscaria.

### CROSS-REFERENCE

Priority is claimed under PCT Art. 8 (1) and Rule 4.10 to U.S. Prov. App. No. 63/446,343, filed Feb. 16, 2023, and incorporated by reference for all purposes as if fully set forth herein.

### FIELD OF THE INVENTION

Provided are improved methods for extracting muscimol from Amanita mushrooms, and more particularly, from Amanita muscaria, including scale-up processes at kilogram scale.

### BACKGROUND OF THE INVENTION

Amanita muscaria, also known as the fly agaric, is a mycorrhizal basidiomycete fungus native to temperate and boreal regions of the Northern Hemisphere. It has also been introduced in countries in the Southern Hemisphere (Guzmán et al. Ann Mus Civ Rovereto. 1998; 14:189-280). A. muscaria is the most iconic and arguably best known of all members of the Amanita genus, which contains some 600 species. Amanita mushrooms have long been a part of the human diet, and the genus contains well-regarded edible species. The genus also contains some of the most deadly poisonous known mushrooms, responsible for about 95% of mushroom fatalities worldwide (Ferenc et al. Med Pr. 2009; 60 (5): 415-426).

Certain Amanita species, with Amanita muscaria prime among them, are also known for their psychoactive properties. Ingestion of the A. muscaria by Siberian tribes of the Kamchatka peninsula and by Indians of the Mexican highlands has been carried out in rituals for centuries. Positive effects were enjoyed by the inhabitants of Siberia and Kamchatka, the Vikings, as well as some North American Indian tribes and the Mayan Guatemalans (Lee et al. J R Coll Physicians Edinb. 2018; 48:85-91). While A. muscaria has been widely used and linked to documented neurotrophic effects on mood, it is nevertheless commonly believed to be a toxic or poisonous mushroom.

A. muscaria is known to contain several psychoactive compounds, such as muscimol. Muscimol is known to cause potentially beneficial anxiolytic, anticonvulsant, amnesic, sedative, hypnotic, euphoriant, and muscle relaxant properties. Muscimol also may have hallucinogenic effects. However, A. muscaria also contains ibotenic acid and muscarine, which may be responsible for deleterious effects of consuming A. muscaria, such as acute toxicity, stomach irritation, nausea, diarrhea, sweating and salivation, lethargy and drowsiness, ataxia, and other somatic symptoms, as well as psychological symptoms such as confusion, euphoria, visual and auditory hallucinations, sensations of floating, distortions of space and time, and retrograde amnesia (Moss et al. Clin Toxicol (Phila). 2019; 57 (2): 99-103). Other components of A. muscaria include heavy metals and other potentially toxic elements and compounds that are absorbed from the soil in the location where a particular mushroom is found growing, and such compounds are therefore variable between specimens depending on where they are collected (Falandysz et al. Environ Sci Pollut Res. 2018; 25:25190-25206).

There is therefore significant value in developing improved formulations and compositions that harness the beneficial properties of A. muscaria and expand access to safe, wellness-promoting products.

### INCORPORATION BY REFERENCE

Each cited patent, publication, and non-patent literature is incorporated by reference in its entirety, as if each was incorporated by reference individually, and as if each is fully set forth herein. No such citation should be construed as an admission that a cited reference comes from an area that is analogous or directly applicable to the invention, nor shall any citation be construed as an admission that a document or underlying information, in any jurisdiction, is prior art or is part of the common general knowledge in the art.

### BRIEF SUMMARY OF THE INVENTION

The following presents a simplified summary of some embodiments of the invention in order to provide a basic understanding thereof. This summary is not an extensive overview of the invention. It is not intended to identify key or critical elements of the invention, or to delineate its full scope. Its sole purpose is to present some embodiments of the invention in a simplified form as a prelude to the more detailed description that is presented later.

In a first aspect, provided is a method for producing a wellness-promoting Amanita muscaria extract from mushroom biomass, comprising: (i) selecting mushroom biomass for extraction; (ii) performing an aqueous extraction of the biomass in the presence of heat to produce the extract; (iii) filtering the extract; (iv) reducing the pH of the extract; (v) heating the extract to reflux; and (vi) collecting the wellness-promoting Amanita muscaria extract.

In embodiments, the biomass is dehydrated prior to performing the aqueous extraction. In embodiments, the biomass is dehydrated to a moisture content of about 1% to about 10%, about 2% to 7%, about 4% to 7%, or about 2%, 3%, or 4% prior to performing the aqueous extraction.

In embodiments, the biomass is ground into a powder prior to performing the aqueous extraction.

In embodiments, selecting the biomass for extraction comprises subjecting the biomass to a quality control analysis. In embodiments, the analysis comprises chemical analysis. In embodiments, the chemical analysis comprises a chromatography technique. In embodiments, the chemical analysis comprises any of high-performance liquid chromatography-mass spectrometry, liquid chromatography-mass spectrometry, and gas chromatography-mass spectrometry.

In embodiments, the chemical analysis comprises determining the concentration of stizolobic acid in the biomass. In embodiments, the biomass is selected for extraction if the concentration of stizolobic acid in the biomass is less than or equal to 100 μg/g. In embodiments, the biomass is selected for extraction if the concentration of stizolobic acid in the biomass is less than or equal to 50 μg/g.

In embodiments, the chemical analysis comprises determining the concentration of stizolobinic acid in the biomass. In embodiments, the biomass is selected for extraction if the concentration of stizolobinic acid in the biomass is less than or equal to 100 μg/g. In embodiments, the biomass is selected for extraction if the concentration of stizolobinic acid in the biomass is less than or equal to 50 μg/g.

In embodiments, the chemical analysis comprises determining the concentration of any of cadmium, arsenic, lead, and mercury in the biomass. In embodiments, the chemical analysis comprises inductively coupled plasma mass spectrometry.

In embodiments, the biomass is selected for extraction if the concentration of cadmium is less than or equal to about 2.0 ppm, 1.5 ppm, 1.0 ppm, or 0.5 ppm. In embodiments, the biomass is selected for extraction if the concentration of cadmium is less than or equal to about 0.5 ppm.

In embodiments, the biomass is selected for extraction if the concentration of arsenic is less than or equal to about 5.0 ppm, 4.0 ppm, 3.0 ppm, 2.0 ppm, 1.5 ppm, 1.0 ppm, or 0.5 ppm. In embodiments, the biomass is selected for extraction if the concentration of arsenic is less than or equal to about 1.5 ppm.

In embodiments, the biomass is selected for extraction if the concentration of lead is less than or equal to about 2.0 ppm, 1.5 ppm, 1.0 ppm, or 0.5 ppm. In embodiments, the biomass is selected for extraction if the concentration of lead is less than or equal to about 0.5 ppm.

In embodiments, the biomass is selected for extraction if the concentration of mercury is less than or equal to about 5.0 ppm, 4.0 ppm, 3.0 ppm, 2.0 ppm, 1.5 ppm, 1.0 ppm, or 0.5 ppm. In embodiments, the biomass is selected for extraction if the concentration of mercury is less than or equal to about 1.5 ppm.

In embodiments, the chemical analysis comprises testing for the presence of Amanita pantherina in the biomass. In embodiments, the biomass is selected for extraction if the biomass comprises less than about 10%, 5%, 4%, 3%, 2%, or 1% (w/w) of Amanita pantherina. In embodiments, the biomass is selected for extraction if the biomass is substantially free of Amanita pantherina.

In embodiments, the chemical analysis comprises determining the concentration of mycotoxins in the biomass. In embodiments, the biomass is selected for extraction if the biomass comprises less than about 20 parts per billion (ppb) of a mycotoxin.

In embodiments, the chemical analysis comprises determining the concentration of pesticides in the biomass. In embodiments, the biomass is selected for extraction if the biomass is substantially free of pesticides.

In embodiments, the analysis comprises an organoleptic analysis. In embodiments, the organoleptic analysis comprises evaluating any of the visual appearance, smell, taste, and texture of the biomass.

In embodiments, the analysis comprises a microbiological analysis. In embodiments, the microbiological analysis comprises any of testing the biomass for the presence of Salmonella spp., testing for the presence of Shiga-toxin producing Escherichia coli (STEC) bacteria, determining the total coliform count, and determining the total aerobic plate count, determining the total yeast and mold count in the biomass.

In embodiments, the aqueous extraction is performed in water heated to between about 70° C. and 100° C. In embodiments, the aqueous extraction is performed in water heated to about 80° C.

In embodiments, the aqueous extraction is performed for between about 15 minutes and about 60 minutes.

In embodiments, filtering the extract comprises passing the extract through a membrane filter. In embodiments, the membrane filter has a pore size of about 200 microns. In embodiments, the membrane filter is a sock filter. In embodiments, filtering the extract comprises passing the extract through two 200-micron sock filters in a parallel configuration.

In embodiments, the pH of the extract is reduced to between about 2.0 and 3.0. In embodiments, the pH of the extract is reduced to between about 2.4 and 2.6. In embodiments, the pH of the extract is reduced using a mineral acid. In embodiments, the mineral acid is hydrochloric acid.

In embodiments, the extract is refluxed at a temperature of between about 95° C. and 100° C. In embodiments, the extract is refluxed for between about 4.25 and 4.75 hours. In embodiments, the extract is refluxed for about 4.5 hours. In embodiments, refluxing the extract reduces the concentration of ibotenic acid and increases the concentration of muscimol in the extract.

In embodiments, the method further comprises increasing the pH of the extract. In embodiments, the pH of the extract is increased to between about 3.5 and 4.5. In embodiments, the pH of the extract is increased to about 4. In embodiments, the pH of the extract is increased by the addition of a hydroxide base. In embodiments, the pH of the extract is increased by the addition of sodium hydroxide pellets.

In embodiments, the weight ratio of muscimol to ibotenic acid in the refluxed extract is between about 10:1 and 200:1. In embodiments, the weight ratio of muscimol to ibotenic acid in the refluxed extract is between about 10:1 and 100:1. In embodiments, the weight ratio of muscimol to ibotenic acid in the refluxed extract is between about 10:1 and 50:1. In embodiments, the weight ratio of muscimol to ibotenic acid in the refluxed extract is between about 10:1 and 25:1. In embodiments, the weight ratio of muscimol to ibotenic acid in the refluxed extract is between about 10:1 and 15:1.

In embodiments, the refluxed extract comprises between about 100 and 130 μg/g of muscimol, between about 7 and 10 μg/g of muscarine, and between about 4 and 12 μg/g of ibotenic acid.

In another aspect, provided is an extract of Amanita muscaria mushroom biomass obtained by the method of any of the disclosed embodiments.

In embodiments, the extract comprises muscimol and ibotenic acid in a weight ratio of between about 10:1 and 200:1. In embodiments, the extract comprises muscimol and ibotenic acid in a weight ratio of between about 10:1 and 100:1. In embodiments, the extract comprises muscimol and ibotenic acid in a weight ratio of between about 10:1 and 50:1. In embodiments, the extract comprises muscimol and ibotenic acid in a weight ratio of between about 10:1 and 25:1. In embodiments, the extract comprises muscimol and ibotenic acid in a weight ratio of between about 10:1 and 15:1.

In embodiments, the extract comprises between about 100 and 130 μg/g of muscimol, between about 7 and 10 μg/g of muscarine, and between about 4 and 12 μg/g of ibotenic acid.

In embodiments, the extract comprises a preservative. In embodiments, the preservative is sodium benzoate, potassium sorbate, or a combination thereof.

In embodiments, the extract comprises a flavoring agent. In embodiments, the flavoring agent is a corn flavor.

In another aspect, provided is a beverage comprising an extract of Amanita muscaria mushroom biomass obtained by the method of any of the disclosed embodiments.

In embodiments, the beverage formulation further comprises a flavoring agent. In embodiments, the flavoring agent is a flavor of plum, prune, date, currant, fig, grape, raisin, cranberry, pineapple, peach, nectarine, banana, apple, pear, guava, apricot, Saskatoon berry, blueberry, plains berry, prairie berry, mulberry, elderberry, Barbados cherry (acerola cherry), choke cherry, chocolate, vanilla, caramel, coconut, olive, raspberry, strawberry, huckleberry, loganberry, dewberry, boysenberry, kiwi, cherry, blackberry, honey dew, green tea, cucumber, quince, buckthorn, passion fruit, sloe, rowan, gooseberry, pomegranate, persimmon, mango, rhubarb, papaya, litchi, lemon, orange, lime, tangerine, mandarin and grapefruit juices, or any combination thereof. In embodiments, the flavoring agent is a mango flavor.

In embodiments, the beverage formulation further comprises magnesium. In embodiments, the beverage formulation further comprises L-theanine.

In another aspect, provided is a dietary supplement comprising an extract of Amanita muscaria mushroom biomass obtained by the method of any of the disclosed embodiments.

In another aspect, provided is an edible formulation comprising an extract of Amanita muscaria mushroom biomass obtained by the method of any of the disclosed embodiments.

In embodiments, the edible formulation is a gummy. In embodiments, the edible formulation is a liquid tincture.

In another aspect, provided is a topical formulation comprising an extract of Amanita muscaria mushroom biomass obtained by the method of any of the disclosed embodiments.

In embodiments, the topical formulation is a skin cream.

In another aspect, provided is a vaporizer formulation comprising an extract of Amanita muscaria mushroom biomass obtained by the method of any of the disclosed embodiments.

These and other objects, features, improvements, and advantages of the invention will be more clearly understood and appreciated from a review of the detailed description of the disclosed embodiments and examples, and by reference to the claims. The foregoing summary is made with the understanding that it is to be considered as a brief and general synopsis of only some of the objects and embodiments disclosed herein, is provided solely for the benefit and convenience of the reader, and is not intended to limit in any manner the scope, or range of equivalents, to which the appended claims are lawfully entitled.

Additional features of the invention are described hereinafter and also form the subject of the claims. It should be appreciated by those in the art that the conception and the disclosed specific methods and structures may be readily utilized as a basis for modifying or designing other structures for carrying out the same purposes of the present disclosure. It should be also realized that such equivalent structures do not depart from the spirit and scope of the invention as set forth in the appended claims.

### BRIEF DESCRIPTION OF THE FIGURE

To further clarify various aspects of the invention, a more particular description is rendered by reference to an exemplary embodiment illustrated in the FIGURE. It will be appreciated that the FIGURE depicts only an illustrated embodiment of the invention and should not be considered limiting of its scope. The FIGURE is merely provided as an exemplary illustration of certain concepts of some embodiments. The FIGURE, and the elements depicted therein, are not necessarily drawn to consistent scale or to any scale. Unless context suggests otherwise, like elements are indicated by like numerals. Certain aspects of the invention are therefore further described and explained with additional specificity and detail, but still by way of example only, with reference to the accompanying FIGURE, which illustrates an exemplary kilogram-scale process for extracting muscimol from A. muscaria according to disclosed embodiments.

### DETAILED DESCRIPTION

Despite the documented benefits of consuming A. muscaria mushrooms, widespread acceptance and adoption by consumers has been limited, in part, by the variability in potency of the mushrooms themselves, as well as concerns about the neurotoxic effects of ibotenic acid, and contamination by heavy metals and other pollutants present in soil. Many of these concerns can be addressed or entirely alleviated by consuming the beneficial components of A. muscaria in the form of an extract, such as the disclosed extracts, which can be standardized to a fixed concentration or a fixed ratio of active agents (e.g., muscimol, muscarine, ibotenic acid) to enable predictable dosing.

The disclosed extracts offer additional advantages, in different embodiments herein: they can have improved shelf stability, they can improve consumer experience (e.g., by providing a more flavorful product), they can be purified and subjected to chemical analysis to ensure they are free of toxins and pollutants, and they can be more easily incorporated into other consumable products, such as edible formulations (e.g., gummies, candies, chocolates, tinctures), topical formulations (e.g., skin creams), and inhalable formulations (e.g., for use in a vaporizer), many such products according to embodiments of the present disclosure.

In cultures in which A. muscaria has a history of entheogenic use, mushrooms are commonly prepared for consumption by boiling them in water to extract the active principles and induce (partial) decarboxylation of ibotenic acid to muscimol (Feeney et al. J Psychoact Drugs. 2011; 42 (4): 4499-506). But, processes suitable for industrial-scale manufacturing are lacking, and there is an unmet need for methods of producing A. muscaria extracts meeting safety and product consistency requirements for commercial sale.

Provided herein are methods for producing an extract of Amanita muscaria mushrooms that meet these needs and others, and having such advantages and improvements as will become readily apparent. Also provided are A. muscaria extracts produced according to these methods, and compositions and formulations of these extracts.

While the invention is described in terms of particular embodiments, examples, and applications, and by reference to the exemplary embodiments depicted in the FIGURE, this description it is not intended to in any way limit its scope to any such embodiments, examples, and applications, and it will be understood that many modifications, substitutions, alternatives, changes, and variations in the described embodiments, examples, applications, and other details of the invention can be made by those skilled in the art without departing from the spirit of the invention, or the scope of the invention described in the claims, including all equivalents to which they are lawfully entitled. While the methods described and illustrated herein may include particular steps, it should be apparent that other methods including fewer, more, or different steps than those described and shown, and methods providing the particular steps in a different order, are also within the spirit and scope of the invention. The methods and uses of any apparatus or system discussed and associated steps shown thus shall be understood as provided for purposes of illustration, not limitation.

### A. GENERAL DEFINITIONS AND TERMS

When introducing elements of the invention or embodiments thereof, the articles “a,” “an,” “the,” and “said” are intended to mean that there are one or more of the elements. Any reference to an element in the singular is therefore not intended to mean “one and only one” unless specifically so stated, but rather “one or more”; therefore, the term “or” standing alone, unless context demands otherwise, shall mean the same as “and/or.” The terms “comprising,” “including,” “such as,” and “having” are also intended to be inclusive and not exclusive (i.e., there may be other elements in addition to the recited elements). Thus, for example, the terms “including,” “may include,” and “include,” as used herein mean, and are used interchangeably with, the phrase “including but not limited to.” The word “exemplary” is used herein to mean “serving as an example, instance, or illustration.” Any aspect, embodiment, process, or implementation described herein as “exemplary” is therefore not to be construed as necessarily preferred or advantageous over others.

Unless otherwise stated, all measurements, values, ratings, positions, magnitudes, sizes, locations, orientations, configurations, and other specifications that are set forth (either expressly or impliedly) in this specification, including in the FIGURE and in the claims that follow, are approximate, and not exact. They are intended to have a reasonable range that is consistent with the functions to which they relate and with what is customary in the art to which they pertain. Unless otherwise stated, all numbers expressing quantities of ingredients, properties such as concentration, reaction conditions, and so forth, used to describe and claim certain embodiments of the invention are to be understood as being modified in some instances by the term “about.” Where the term “about” is used to modify a value, it means that a range of values that includes the specified value is also encompassed, which a person of ordinary skill would consider reasonably similar to the specified value. Where numerical values may be modified by the term “about,” it will be understood that in some embodiments (equivalently herein, and simply for shorthand, “in embodiments”), such values are modified by “about,” but in other embodiments, such values are within a further degree of precision. In embodiments, “about” means within a standard deviation using measurements generally acceptable in the art. In some embodiments “about” means within manufacturing tolerances. In other embodiments, “about” means a range extending to +10% of the specified value. The numerical parameters set forth in the written description and attached claims are approximations that can vary depending upon the desired properties sought to be obtained by a particular embodiment. The term “substantially,” when applied to modify a parameter or characteristic herein, will be read in the context of the invention and in light of the knowledge in the art to provide certainty, e.g., by using a standard that is recognized in the art for measuring the meaning of substantially as a term of degree, or by ascertaining the scope as would one of skill. For example, the term “substantially free” is used in embodiments to refer to an amount of a substance that is below the Level of Detection (LOD) or Level of Quantification (LOQ) for conventional methods used to determine the amount or concentration of that substance.

In embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable, taking into consideration manufacturing tolerances, for example. The numerical values presented in some embodiments of the invention may contain certain errors necessarily resulting from the standard deviation found in their respective testing measurements.

Generally, the nomenclature used and procedures performed herein are those known in field(s) relating to that of one or more aspects of the invention, and are those that will be well-known and commonly employed in such field(s). Standard techniques and procedures will be those generally performed according to conventional methods in the art. Unless defined otherwise, all technical and scientific terms herein have the meaning as commonly understood by a person having ordinary skill in the art to which this invention belongs, who as a shorthand may be referred to simply as “one of skill.”

Further definitions that may assist the reader in understanding the disclosed embodiments are as follows; however, it will be appreciated that they will not be used to limit the scope of the invention, which shall be properly interpreted and understood by reference to the full specification (as well as any plain meaning known to one of skill) in view of the language used in the claims. In general, the terminology used herein is for the purpose of describing particular embodiments only and is not intended to be limiting.

“Amanita muscaria” will be understood to include the Amanita muscaria var. Muscaria (Euro-Asian fly agaric), Amanita muscaria var. flavivolvata (American fly agaric), Amanita muscaria var. guessowii (American fly agaric, yellow variant), and Amanita muscaria var. inzengae (“Inzenga's” fly agaric); also within the scope and spirit of the invention, and which shall be considered within the definition of A. muscaria or as equivalents thereof, are such other ibotenic acid and muscimol-containing Amanita species that are not known to be deadly poisonous, as will be readily appreciated as within the general knowledge in the art.

“Cap” refers to the pileus of a mushroom, including any pieces of universal veil attached thereto, e.g., as warts or irregular patches. Generally the cap of an individual Amanita mushroom can be easily separated by hand or other mechanical means from its “stipe” or stem. The term “mushroom biomass” (which may be used interchangeably with “mushroom” or “mushrooms”) encompasses the caps, stipes, and any other structure that is present at any stage of an Amanita mushroom's development, whether usually found above ground, underground, or contained within a biosynthetic production system. Such structures include, but are not limited to, fruiting bodies, sclerotia, protoplasts, spores, and mycelium.

“Ibotenic acid” means(S)-2-amino-2-(3-hydroxyisoxazol-5-yl) acetic acid.

“Muscimol” means 5-(aminomethyl)-1,2-oxazol-3 (2H)-one.

“Muscarine” means 2,5-anhydro-1,4,6-trideoxy-6-(trimethylazaniumyl)-D-ribo-hexitol.

“Decarboxylation” refers to a chemical reaction that removes a carboxyl group and releases carbon dioxide (CO₂), thereby replacing a carboxyl group (—COOH) with a hydrogen atom (H); e.g., as RCO₂H→RH+CO₂. Ibotenic acid is converted to muscimol by decarboxylation:

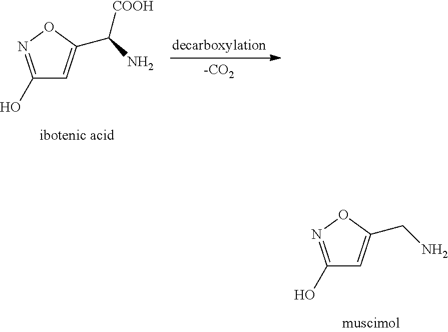 [SMILES: N[C@H](C(=O)O)c1cc(O)no1 ; NCc1cc(O)no1]

Still additional definitions and abbreviations are provided elsewhere herein.

### B. EXTRACTION PROCESSES

a. Selection and Preparation of Mushrooms

In accordance with embodiments of the invention, processes are disclosed for extracting muscimol from A. muscaria. In some aspects are improved processes for extraction at scale, such as at kilogram scale.

One example embodiment of the invention is provided in Example 1, with reference to the FIGURE.

In embodiments, A. muscaria mushrooms (for shorthand, also sometimes “mushrooms” herein) that have been harvested are inspected to ensure that they are of suitable quality. Such inspection may identify debris, dirt, and other organic matter (which will be discarded), may be useful in confirming the correct species, and removing mistaken species, especially those that are toxic or poisonous; and/or may aid in determining general quality, including overall appearance, cleanliness, color, damage, apparent age, presence or absence of signs of worms or insects, etc. Organoleptic analysis, such as visual inspection and tactile inspection, may be utilized. FIGS. 8 and 9 of WO2022/187974A1, the entirety of which is incorporated by reference as if fully set forth herein, are photographic examples of high and low quality mushroom caps.

High quality mushroom caps may be selected, for example, based on whether they have retained their natural color, distinct patterns, and shape. Low quality mushroom caps may be selected, for example, based on whether they are visually desiccated, partially decomposed, moldy, include wormholes, are of varying shades of brown, and/or have lost their original shape.

In embodiments, the harvested mushrooms are mature. In embodiments, the harvested mushrooms are not yet mature. In embodiments, mushrooms are not harvested from areas subject to industrial pollution or nuclear fallout.

In embodiments, the mushrooms are frozen for a period of storage prior to further processing, upon which time the mushrooms are thawed.

In embodiments, where the mushrooms comprise both a cap and at least a partial stipe, the mushroom caps are removed from the stipe. In some such embodiments, mushroom caps alone are selected for extraction. In embodiments, mushroom caps alone are selected for extraction because they have the highest concentration of target compounds, and lead to higher overall yields of muscimol.

In embodiments, the mushrooms are dried until they are sufficiently dry, for example until they have a moisture content from about 2% to about 3% moisture by weight. In other embodiments, the mushrooms have less than 2% or more than 3% moisture by weight, for example, a moisture content of about 4% to about 7% by weight. In embodiments, the mushrooms have a moisture content from about 1% to about 7%. In embodiments, the mushrooms have a moisture content greater than 7%. If not sufficiently dry when first inspected, the mushrooms may be dried or dehydrated. Mushrooms may be dried or dehydrated such as in a dehydrator or by application of heat using a method including conduction, convection, or radiation. In embodiments, the mushrooms are not dried or dehydrated. For example, in embodiments, the mushrooms may be processed fresh, or may be processed without further drying.

In embodiments, if the mushrooms are dried below 2%, or at too high of a temperature, muscimol and ibotenic acid may be degraded, reducing the final yield of muscimol that will be obtained by a disclosed process. In some embodiments an appropriate amount of drying or level of moisture content is determined. In embodiments, the appropriate level of moisture content is determined by whether the mushrooms easily snap in half or in parts. In embodiments, the mushrooms that easily snap in half or in parts are in the desired moisture range. In embodiments, dehydration is performed until the mushrooms are what is known in the art as “cracker dry.” In embodiments, dehydration is performed until the mushrooms are suitably able to be ground into a powder, such as using a disclosed grinding means.

In embodiments, drying the harvested mushrooms is an optional step, as discussed further below. Therefore in some embodiments, the mushrooms are not dried, or have a moisture content above 7%.

In some embodiments wherein drying is performed, drying may be completed at a maximum temperature of 50° C., including 49° C., 48° C., 47° C., 46° C., 45° C., 44° C., 43° C., 42° C., 41° C., 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., or a temperature less than 20° C. In embodiments, the mushrooms are dried in a circulated air dehydration unit, a forced air food dryer, or any other such device capable of heating and/or dehydrating the mushrooms by conduction, convection, and/or radiation.

Additionally, drying may, in some embodiments, last from about 24 hours to about 48 hours, including 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, and 48 hours, wherein the range is inclusive and each value will be understood as being both modified and not modified by the term “about.” Likewise, in embodiments, heating may be completed for less than 24 hours, such as 20 hours, 16 hours, 12 hours, 8 hours, or less than 8 hours; or greater than 48 hours, such as 56 hours, 60 hours, 65 hours, 72 hours, or 80 hours (inclusive).

In embodiments, whether or not the mushrooms are dehydrated, they may be sealed in plastic bags or airtight containers and stored in darkness until processing. In embodiments, the temperature at which the mushrooms are stored corresponds with how soon processing may take place. Generally, mushrooms are stored at between about −25° C. and about 3° C., with storage at higher temperatures if processing will more imminently occur (such as within about 5 days), and at lower temperatures if processing will be further in the future. Higher temperatures, and in particular temperatures above 3° C., may result in a loss of potency and gradual destruction of the mushrooms by macroscopic and microscopic organisms.

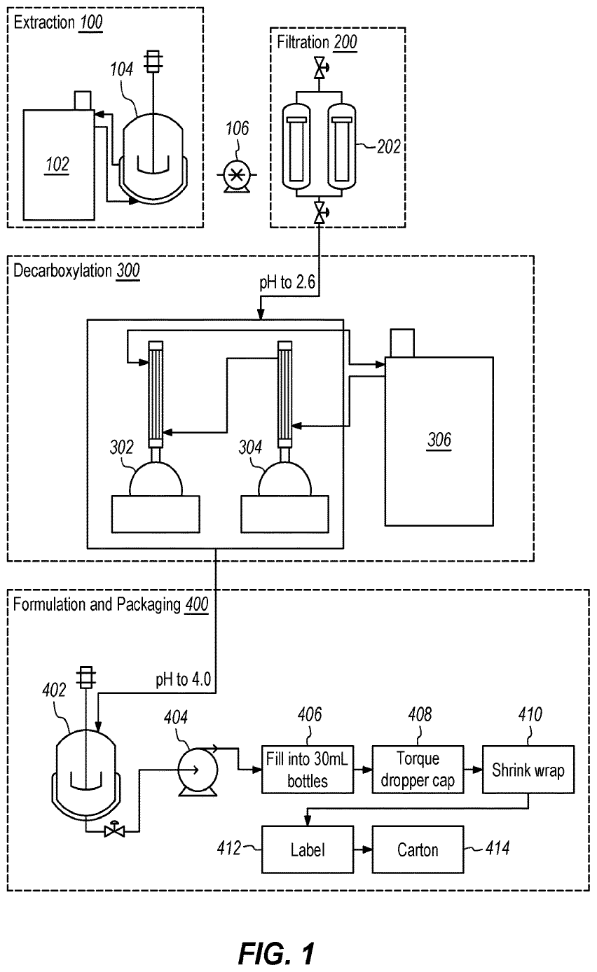

In embodiments, the mushrooms are ground. The mushrooms may be ground to a powder, such as a fine or a substantially fine powder, for example by an industrial scale food processor, coffee grinder, blender, or such device, and grinding may take place for about 10 seconds, 20 seconds, 30 seconds, 45 seconds, 1 minute, 2 minutes, 3 minutes, 4 minutes, or about 5 minutes, for example. One of skill will appreciate how to obtain powder of an appropriate size; see, for example, the visual representations provided in FIGS. 2 and 3 of WO2022/187974A1, incorporated by reference as if fully set forth herein.

In embodiments, the mushrooms are subjected to one or more quality control analyses. In embodiments, a quality control analysis is performed prior to grinding. In other embodiments, a quality control analysis is performed after grinding. For example, in embodiments, a batch of mushrooms or ground mushroom powder may be analyzed to determine whether the levels of muscimol, muscarine, and ibotenic acid are within specifications. Although certain embodiments herein refer to subjecting ground mushroom powder to an analysis, this will be understood to be merely for exemplary purposes; such embodiments shall therefore be understood to be equivalently applicable to fresh mushrooms or any other form of mushroom biomass. In embodiments, a batch of mushrooms or ground powder may be analyzed to determine whether heavy metal content and/or pesticide content is within specifications. Specifications may include safety, process, and/or production specifications. An analysis may be performed, for example, by high performance liquid chromatography tandem mass spectrometry (“HPLC-MS/MS”). As one example, in embodiments, a batch must be determined to have at least 500 micrograms per gram of muscimol to pass quality control.

In embodiments, the quality control analysis comprises a chemical analysis. In embodiments, a batch of mushrooms or ground mushroom powder is subjected to chemical analysis to confirm the purity of the constituent mushroom species.

In embodiments, the chemical analysis comprises determining the concentrations of a heavy metal in the ground mushroom powder, e.g., by using inductively coupled plasma mass spectrometry (ICP-MS). In embodiments, the chemical analysis comprises determining the concentrations of any of cadmium, arsenic, lead, and mercury. In embodiments, if ground powder is determined by chemical analysis to contain substantial amounts of any of cadmium, arsenic, lead, and mercury, it is not used in disclosed methods. In embodiments, the concentration of cadmium in the ground mushroom powder used in a disclosed method is less than about 2.0 ppm, 1.5 ppm, 1.0 ppm, or 0.5 ppm. In embodiments, the concentration of cadmium in the ground mushroom powder used in a disclosed method is less than about 0.5 ppm. In embodiments, the concentration of arsenic in the ground mushroom powder used in a disclosed method is less than about 5.0 ppm, 4.0 ppm, 3.0 ppm, 2.0 ppm, 1.5 ppm, 1.0 ppm, or 0.5 ppm. In embodiments, the concentration of arsenic in the ground mushroom powder used in a disclosed method is less than about 1.5 ppm. In embodiments, the concentration of lead in the ground mushroom powder used in a disclosed method is less than about 2.0 ppm, 1.5 ppm, 1.0 ppm, or 0.5 ppm. In embodiments, the concentration of lead in the ground mushroom powder used in a disclosed method is less than about 0.5 ppm. In embodiments, the concentration of mercury in the ground mushroom powder used in a disclosed method is less than about 5.0 ppm, 4.0 ppm, 3.0 ppm, 2.0 ppm, 1.5 ppm, 1.0 ppm, or 0.5 ppm. In embodiments, the concentration of mercury in the ground mushroom powder used in a disclosed method is less than about 1.5 ppm.

In embodiments, the chemical analysis comprises determining the concentrations of a mycotoxin in the ground mushroom powder. Suitable techniques for determining mycotoxin concentrations include, for example, chromatography (e.g., liquid or gas chromatography) coupled with mass spectrometry. In embodiments, a disclosed method comprises determining by chemical analysis the concentrations of any of aflatoxin B1, aflatoxin B2, aflatoxin G1, aflatoxin G2, and ochratoxin. Exemplary cutoff values for maximum acceptable mycotoxin concentrations in a ground mushroom sample used in a disclosed method are: less than about 20 ppb of the total amount of aflatoxins (e.g., aflatoxin B1+aflatoxin B2+aflatoxin G1+aflatoxin G2); less than about 5 ppb of aflatoxin B1; and less than about 5 ppb of ochratoxin.

In embodiments, if a ground mushroom sample comprises less than about 100 ppb of the total concentration of aflatoxins (e.g., aflatoxin B1+aflatoxin B2+aflatoxin G1+aflatoxin G2), the mushroom biomass is selected for extraction. In embodiments, the total concentration of aflatoxins in the mushroom biomass selected for extraction is less than about 50 ppb. In embodiments, the total concentration of aflatoxins in the mushroom biomass selected for extraction is less than about 40 ppb. In embodiments, the total concentration of aflatoxins in the mushroom biomass selected for extraction is less than about 30 ppb. In embodiments, the total concentration of aflatoxins in the mushroom biomass selected for extraction is less than about 20 ppb. In embodiments, the total concentration of aflatoxins in the mushroom biomass selected for extraction is less than about 10 ppb.

In embodiments, if a ground mushroom sample comprises less than about 20 ppb of aflatoxin B1, the mushroom biomass is selected for extraction. In embodiments, the concentration of aflatoxin B1 in the mushroom biomass selected for extraction is less than about 10 ppb. In embodiments, the concentration of aflatoxin B1 in the mushroom biomass selected for extraction is less than about 5 ppb.

In embodiments, if a ground mushroom sample comprises less than about 20 ppb of ochratoxin, the mushroom biomass is selected for extraction. In embodiments, the concentration of ochratoxin in the mushroom biomass selected for extraction is less than about 10 ppb. In embodiments, the concentration of ochratoxin in the mushroom biomass selected for extraction is less than about 5 ppb.

In embodiments, the chemical analysis comprises determining the concentrations of a pesticide in the ground mushroom powder. Suitable techniques for determining pesticide concentrations include, for example, chromatography (e.g., liquid or gas chromatography) coupled with mass spectrometry. In embodiments, a disclosed method comprises determining by chemical analysis the concentrations of any of abamectin (e.g., avermectin B1a), acequinocyl, acetamiprid, aldicarb, allethrin, atrazine, azadirachtin, azoxystrobin, benzovindiflupyr, bifenazate, bifenthrin, boscalid, buprofezin, carbaryl, carbofuran, chlorantraniliprole, chlorphenapyr, chlorpyrifos, clofentezine, clothianidin, fenhexamid, fenoxycarb, fenpyroximate, fensulfothion, fenthion, fenvalerate, fipronil, flonicamid, fludioxonil, fluopyram, hexythiazox, imazalil, coumaphos, cyfluthrin, cypermethrin, cyprodinil, daminozide, deltamethrin, diazinon, dichlorvos, dimethoate, dimethomorph, dinotefuran, diuron, dodemorph, endosulfan sulfate, endosulfan-alpha, endosulfan-beta, ethoprophos, etofenprox, etoxazole, etridiazole, myclobutanil, naled, novaluron, oxamyl, paclobutrazol, parathion-methyl, permethrin. phenothrin, phosmet, piperonyl butoxide, pirimicarb, prallethrin, imidacloprid, iprodione, kinoprene, kresoxim-methyl, (lambda) cyhalothrin, malathion, metalaxyl, methiocarb, methomyl, methoprene, mevinphos, MGK-264, spiromesifen, spirotetramat, spiroxamine, tebuconazole, tebufenozide, teflubenzuron, tetrachlorvinphos, propiconazole, propoxur, pyraclostrobin, pyrethrins, pyridaben, pyriproxyfen, quintozene, resmethrin, spinetoram, spinosad, spirodiclofen, tetramethrin, thiabendazole, thiacloprid, thiamethoxam, thiophanate-methyl, trifloxystrobin, and trifloxystrobin. The Level of Quantification (LOQ) for a specific pesticide and specific technique can vary, however, these typically range from about 0.1 to 2.0 ppm for most liquid or gas chromatography-mass spectrometry techniques. In embodiments, if a ground mushroom sample has a quantifiable amount of a pesticide (i.e., a concentration exceeding the LOQ), it is not used in disclosed methods. In embodiments, if a ground mushroom sample is substantially free of pesticides (e.g., pesticide concentrations are below the LOQ), the mushroom biomass is selected for extraction.

In embodiments, a batch of mushrooms or ground mushroom powder is subjected to chemical analysis to confirm the identity of the constituent mushroom species. For example, in some embodiments chemical means is used to confirm that the mushrooms or ground powder are or are from Amanita muscaria, as opposed to one or more other fungal species (which could in principle be present due to contamination, or misidentification during harvesting). In embodiments, the chemical analysis is a chromatographic method, such as liquid chromatography (LC), gas chromatography (GC), high-performance liquid chromatography (HPLC). The chemical analysis may comprise mass spectrometry (MS), optionally coupled with a chromatography method, such as GC-MS, LC-MS, or HPLC-MS. In embodiments, the chemical analysis is performed by HPLC-MS.

In embodiments, the chemical analysis comprises determining the concentration of at least one amino acid that exists at a characteristic concentration or in a characteristic ratio to another chemical component (such as another amino acid), thereby providing a “chemical fingerprint” that can be used to confirm the identity and/or purity of the ground powder. Example 2 provides an exemplary procedure for chemical fingerprinting of ground mushroom powder according to disclosed embodiments. In embodiments, the amino acid is stizolobic acid. In embodiments, the amino acid is stizolobinic acid. In embodiments, the chemical analysis comprises determining the concentrations of both stizolobic acid and stizolobinic acid in the ground powder. In embodiments, the chemical analysis comprises subjecting the ground powder to HPLC-MS to determine the concentrations of both stizolobic acid and stizolobinic acid in the ground powder. Stizolobic acid and stizolobinic acid are found in all Amanita species, but are present in different concentrations in Amanita muscaria compared to, for example, Amanita pantherina. For example, Amanita muscaria typically contains less than about 100 μg or less than about 50 μg of stizolobic acid, stizolobinic acid, or the sum fo both acids, per gram of dried mushroom powder. On the other hand, Amanita pantherina can contain greater than 400 μg of stizolobic acid, stizolobinic acid, or the sum fo both acids, per gram of dried mushroom powder. Therefore, determining the concentrations of stizolobic acid and stizolobinic acid can allow for chemical fingerprinting of Amanita muscaria vs Amanita pantherina to identify whether the ground mushroom powder contains Amanita pantherina, which in some embodiments is undesired. In embodiments, if ground powder is determined by chemical analysis to contain Amanita pantherina, it is not used in disclosed methods.

In embodiments, the biomass is selected for extraction if the biomass comprises less than about 10%, 5%, 4%, 3%, 2%, or 1% (w/w) of Amanita pantherina. In embodiments, the biomass is selected for extraction if the biomass comprises less than about 10% (w/w) of Amanita pantherina. In embodiments, the biomass is selected for extraction if the biomass comprises less than about 5% (w/w) of Amanita pantherina. In embodiments, the biomass is selected for extraction if the biomass comprises less than about 4% (w/w) of Amanita pantherina. In embodiments, the biomass is selected for extraction if the biomass comprises less than about 3% (w/w) of Amanita pantherina. In embodiments, the biomass is selected for extraction if the biomass comprises less than about 2% (w/w) of Amanita pantherina. In embodiments, the biomass is selected for extraction if the biomass comprises less than about 1% (w/w) of Amanita pantherina. In embodiments, the biomass is selected for extraction if the biomass is substantially free of Amanita pantherina.

In embodiments, a batch of mushrooms or ground mushroom powder is subjected to microbiological analysis to confirm the safety of the resulting extracts for human consumption. In embodiments, the microbiological analysis comprises testing for the presence of Salmonella spp. in the ground mushroom powder (e.g., using the AOAC 2016.01 method). In embodiments, ground mushroom powder will not be used in a disclosed method if any amount of Salmonella spp. is detected in a sample of ground mushroom powder of predetermined weight (e.g., 25 g). In embodiments, the microbiological analysis comprises testing for the presence of Shiga-toxin producing Escherichia coli (STEC) bacteria in the ground mushroom powder (e.g., using the 3M MDS STEC/EAE method). In embodiments, ground mushroom powder will not be used in a disclosed method if any amount of STEC bacteria is detected in a sample of ground mushroom powder of predetermined weight (e.g., 25 g). In embodiments, the microbiological analysis comprises determining the total coliform count in a sample of ground mushroom powder (e.g., using the FDA BAM-ECC AGAR method). In embodiments, ground mushroom powder will not be used in a disclosed method if the total coliform count is greater than about 10² cfu/g. In embodiments, the microbiological analysis comprises determining the total aerobic plate count in a sample of ground mushroom powder (e.g., using the FDA BAM method). In embodiments, ground mushroom powder will not be used in a disclosed method if the total aerobic plate count is greater than about 10² cfu/g. In embodiments, the microbiological analysis comprises determining the total yeast and mold count in a sample of ground mushroom powder (e.g., using the FDA BAM method). In embodiments, ground mushroom powder will not be used in a disclosed method if the total yeast and mold count is greater than about 10² cfu/g.

In embodiments, the quality control analysis comprises a chemical analysis and an organoleptic analysis. In embodiments, the quality control analysis comprises a chemical analysis and a microbiological analysis. In embodiments, the quality control analysis comprises a microbiological analysis and an organoleptic analysis. In embodiments, the quality control analysis comprises a chemical analysis, an organoleptic analysis, and a microbiological analysis.

In embodiments, the quality control analysis comprises determining the concentration of stizolobic acid and heavy metals in the mushroom biomass. In embodiments, the quality control analysis comprises determining the concentrations of stizolobic acid and mycotoxins in the mushroom biomass. In embodiments, the quality control analysis comprises determining the concentrations of stizolobic acid and pesticides in the mushroom biomass. In embodiments, the quality control analysis comprises determining the concentration of stizolobic acid in the mushroom biomass, and conducting a microbiological analysis on the mushroom biomass. In embodiments, the quality control analysis comprises determining the concentration of stizolobic acid in the mushroom biomass, and conducting an organoleptic analysis on the mushroom biomass.

In embodiments, the quality control analysis comprises determining the concentration of stizolobinic acid and heavy metals in the mushroom biomass. In embodiments, the quality control analysis comprises determining the concentrations of stizolobinic acid and mycotoxins in the mushroom biomass. In embodiments, the quality control analysis comprises determining the concentrations of stizolobinic acid and pesticides in the mushroom biomass. In embodiments, the quality control analysis comprises determining the concentration of stizolobinic acid in the mushroom biomass, and conducting a microbiological analysis on the mushroom biomass. In embodiments, the quality control analysis comprises determining the concentration of stizolobinic acid in the mushroom biomass, and conducting an organoleptic analysis on the mushroom biomass.

In embodiments, the quality control analysis comprises determining the concentration of both stizolobic acid and stizolobinic acid and heavy metals in the mushroom biomass. In embodiments, the quality control analysis comprises determining the concentrations of both stizolobic acid and stizolobinic acid and mycotoxins in the mushroom biomass. In embodiments, the quality control analysis comprises determining the concentrations of both stizolobic acid and stizolobinic acid and pesticides in the mushroom biomass. In embodiments, the quality control analysis comprises determining the concentration of both stizolobic acid and stizolobinic acid in the mushroom biomass, and conducting a microbiological analysis on the mushroom biomass. In embodiments, the quality control analysis comprises determining the concentration of both stizolobic acid and stizolobinic acid in the mushroom biomass, and conducting an organoleptic analysis on the mushroom biomass.

In embodiments, the quality control analysis comprises determining the concentrations of heavy metals and mycotoxins in the mushroom biomass. In embodiments, the quality control analysis comprises determining the concentrations of heavy metals and pesticides in the mushroom biomass. In embodiments, the quality control analysis comprises determining the concentrations of heavy metals and in the mushroom biomass, and conducting a microbiological analysis on the mushroom biomass. In embodiments, the quality control analysis comprises determining the concentrations of heavy metals and in the mushroom biomass, and conducting an organoleptic analysis on the mushroom biomass.

In embodiments, the quality control analysis comprises determining the concentrations of pesticides and mycotoxins in the mushroom biomass. In embodiments, the quality control analysis comprises determining the concentrations of pesticides and in the mushroom biomass, and conducting a microbiological analysis on the mushroom biomass. In embodiments, the quality control analysis comprises determining the concentrations of pesticides and in the mushroom biomass, and conducting an organoleptic analysis on the mushroom biomass.

In embodiments, the quality control analysis comprises determining the concentrations of mycotoxins and in the mushroom biomass, and conducting a microbiological analysis on the mushroom biomass. In embodiments, the quality control analysis comprises determining the concentrations of mycotoxins and in the mushroom biomass, and conducting an organoleptic analysis on the mushroom biomass.

b. Initial Extraction

Water (which, in some embodiments, is distilled water) is then obtained. In embodiments, the pH is determined, and if not about 7, replaced or adjusted as needed to obtain water having a pH of about 7. In embodiments, the pH of the water is between about 6.9 and 7.1 In embodiments, the water is boiled before use, which can remove impurities from the water.

The mushrooms are then placed in the water and stirred, ultimately yielding an A. muscaria extract. In one example, a 110 L jacketed tank fitted with a mechanical overhead stirrer is used to stir the water/mushroom mixture. However, this is merely an exemplary, non-limiting embodiment. In other embodiments, another vessel contains the water/mushroom mixture, which may optionally further comprise a means for stirring. In embodiments, the vessel is heated directly (e.g., by direct firing with an open flame under the container, or by means of electric heating elements incorporated into the container's design). In other embodiments, heating is accomplished using an external heater that circulates a hot fluid (e.g., glycol) or vapor (e.g., steam) through the external “jacket” or “sleeve” of a jacketed vessel.

The temperature of the mixture of water and ground mushrooms is maintained at a temperature of about 70° C. to about 100° C., including about 70° C., 75° C., 80° C., 85° C., 90° C., 95° C., or 100° C. In embodiments, the temperature is maintained at about 80° C.

In embodiments, the mixture is stirred for between about 5 minutes and 60 minutes, such as for 5 minutes, 10 minutes, 15 minutes, 20 minutes, 20 minutes, 30 minutes, 40 minutes, 50 minutes, or 60 minutes. In embodiments, the mixture is stirred for between about 15 minutes and 60 minutes. In other embodiments, the mixture is stirred for less than 5 minutes, or more than 60 minutes. In embodiments, stirring is at between 700 rpm and 2500 rpm, including about 750 rpm, 800 rpm, 850 rpm, 900 rpm, 950 rpm, 1000 rpm, 1050 rpm, 1100 rpm, 1150 rpm, 1200 rpm, 1250 rpm, 1300 rpm, 1350 rpm, 1400 rpm, 1450 rpm, 1500 rpm, 1550 rpm, 1600 rpm, 1650 rpm, 1700 rpm, 1750 rpm, 1800 rpm, 1850 rpm, 1900 rpm, 1950 rpm, 2000 rpm, 2050 rpm, 2100 rpm, 2150 rpm, 2200 rpm, 2250 rpm, 2300 rpm, 2350 rpm, 2400 rpm, 2450 rpm, and 2500 rpm. In embodiments, stirring is at less than 700 rpm, or at more than 2500 rpm.

The ratio of water to ground mushrooms can have impacts on the ease and efficiency of the extraction process. For example, if the ratio of water to ground mushrooms is too low (i.e., there is not much water relative to the amount of ground mushrooms), the viscosity of the mixture can become high enough to impede stirring. In such cases, the high viscosity can reduce the extraction efficiency and/or present processing challenges. For example, if the water/mushroom mixture is too thick, it may not be possible to effectively filter the mixture to remove the mushroom biomass. In embodiments, the ratio of water to mushroom may be from about 5 L of water per 1 kg of mushroom (i.e., a 5:1 water:mushroom ratio), up to about 20 L of water per 1 kg of mushroom (i.e., a 20:1 water:mushroom ratio). In embodiments, the water:mushroom ratio is about 5:1, 10:1, 11:1, 12:1, 13:1, 14:1, 15:1, 16:1, 17:1, 18:1, 19:1 or 20:1, or values in between these ratios. In embodiments, the water:mushroom ratio is about 14:1, e.g., about 14 L of water for each 1 kg of mushroom.

c. Filtration

In embodiments, the extract is filtered to remove solids, such as particles of ground mushroom. Transferring the extract from the initial extraction tank to the filtration apparatus may be conducted by any suitable means known to those of skill in the art. In one example, a diaphragm pump is used to transfer the extract from the initial extraction tank to the filtration apparatus. The extract can be filtered while still hot from the initial extraction, or it can be cooled prior to filtration. In embodiments, the extract is filtered while still hot from the initial extraction.

Many filters known to those of skill may be utilized, with preference to filters capable of filtering out both large and small particles. In embodiments, a plurality of filters are used in series, wherein the filter pore size decreases with each additional filter. In such embodiments, at least two, at least three, at least four, at least five, or more than five filters are utilized in series. In embodiments, one or more filters of a specific mesh or pore size are used, for example where certain particles of a given size are desired to be removed and/or to be retained in the extract, and/or to be individually isolated.

In embodiments, the filter is a membrane filter (e.g., a sock filter). In embodiments, the membrane filter has a pore size of about 50 microns to about 500 microns. In embodiments, the membrane filter has a pore size of about 50 microns, 75 microns, 100 microns, 150 microns, 200 microns, 250 microns, 300 microns, or 500 microns. In embodiments, the membrane filter has a pore size of about 200 microns.

In embodiments, a plurality of filters may be used in a parallel arrangement, wherein the filter pore size is the same for each filter. In such embodiments, at least two, at least three, at least four, at least five, or more than five filters may be utilized in parallel. This may be particularly useful in embodiments where it is desired to filter a large volume of liquid. In embodiments, a plurality of 200-micron membrane filters are used in parallel. For example, in embodiments, two 200-micron sock filters are used in parallel.

In embodiments, pressure is applied during the filtration process, such as with use of mechanical pressing, compressed gas, or any suitable means of applying pressure to the feed side of a filter or filtering apparatus or device. In embodiments, the extract is drawn through the filter(s) by vacuum filtration. In embodiments, the filtered extract (i.e., the filtrate) is also centrifuged to separate particulate matter and a supernatant.

d. Decarboxylation

The pH of the extract is reduced by the addition of a suitable acid. Without being bound by theory, the acidic environment causes ibotenic acid to convert into muscimol by a decarboxylation reaction. If the mushrooms are not sufficiently dried (to between about 2% to about 3% moisture content), a higher concentration of HCl or other acid may be needed to achieve the same degree of conversion of ibotenic acid to muscimol.

In embodiments, the pH is reduced from the initial pH of the extract, which may be about 7 (the pH of distilled water) to a pH of about 1.0 to about 4.0. In embodiments, the pH of the extract is reduced to about 1.0, to about 1.5, to about 2.0, to about 2.5, to about 3.0, to about 3.5, or to about 4.0, wherein the range is inclusive. In embodiments, the pH of the extract is reduced to between about 2 and 3. In embodiments, the pH of the extract is reduced to between about 2.4 and 2.6. In embodiments, the pH of the extract is reduced to about 2.6.

In embodiments, the acid is a mineral acid. In embodiments, the acid is hydrochloric acid (HCl), hydrofluoric acid (HF), hydrobromic acid (HBr), hydroiodic acid (HI), nitric acid, phosphoric acid, sulfuric acid, boric acid, or carbonic acid. In embodiments, the acid is an organic acid. In embodiments, the acid is acetic acid, citric acid, or oxalic acid. The acid can alternatively be another known compound capable of reducing the pH of the extract.

In embodiments, the extract is heated to reflux. In embodiments, the temperature of extract during the reflux is between about 95° C. and about 105° C. In embodiments, the temperature of the extract during the reflux is about 95° C., 96° C., 97° C., 98° C., 99° C., 100° C., 101° C., 102° C., 103° C., 104° C., or 105° C. In embodiments, the temperature of the extract during the reflux is about 99° C.

In embodiments, the duration of the reflux is about 1 to 6 hours. For example, the duration of the reflux may be about 1 hour, 2 hours, 3 hours, 4 hours, 5 hours, or 6 hours, or times in between these values. In embodiments, the duration of the reflux is between about 2 hours and 6 hours. In embodiments, the duration of the reflux is between about 4 hours and 5 hours. In embodiments, the duration of the reflux is about 4.5 hours.

Reflux may be performed in any vessel that can be heated to the reflux temperature. In embodiments, the container is a flask, such as a glass flask, or a glass round-bottom flask. In embodiments, the container has a capacity of about 5 L to about 20 L. In some specific embodiments, the container has a capacity of about 8 L, or about 12 L. In embodiments, the container may be fitted with a reflux condenser, such as a glass or Pyrex Graham condenser having a coolant-jacketed spiral coil running the length of the condenser serving as the vapor-condensate path. In embodiments, the condenser is coupled to a means for chilling, such as a water pump that circulates chilled water through the coolant jacket.

The container may likewise be heated by any suitable means known to the skilled artisan, including by direct flame or through the use of an additional heating apparatus, such as an electric heating mantle, sand bath, steam bath, oil bath, or hot plate. In one specific embodiment, an 8 L flask containing the extract is heated using a heating mantle with a 10 L capacity. In another specific embodiment, a 10 L flask containing the extract is heated using a heating mantle with a 12 L capacity.

In embodiments, a plurality of containers are used in a parallel arrangement to enable simultaneous refluxing of different portions of the total volume of the extract, such as to reflux a large volume of liquid.

In embodiments, the weight ratio of muscimol to ibotenic acid in the refluxed extract (i.e., post-decarboxylation) is between about 10:1 and 1000:1. In embodiments, the weight ratio of muscimol to ibotenic acid in the refluxed extract is between about 10:1 and 500:1. In embodiments, the weight ratio of muscimol to ibotenic acid in the refluxed extract is between about 10:1 and 400:1. In embodiments, the weight ratio of muscimol to ibotenic acid in the refluxed extract is between about 10:1 and 300:1. In embodiments, the weight ratio of muscimol to ibotenic acid in the refluxed extract is between about 10:1 and 200:1. In embodiments, the weight ratio of muscimol to ibotenic acid in the refluxed extract is between about 10:1 and 100:1. In embodiments, the weight ratio of muscimol to ibotenic acid in the refluxed extract is between about 10:1 and 50:1. In embodiments, the weight ratio of muscimol to ibotenic acid in the refluxed extract is between about 10:1 and 40:1. In embodiments, the weight ratio of muscimol to ibotenic acid in the refluxed extract is between about 10:1 and 30:1. In embodiments, the weight ratio of muscimol to ibotenic acid in the refluxed extract is between about 10:1 and 20:1. In embodiments, the weight ratio of muscimol to ibotenic acid in the refluxed extract is between about 10:1 and 15:1.

In embodiments, the refluxed extract comprises between about 50 and 500 μg/g of muscimol. In embodiments, the refluxed extract comprises between about 50 and 400 μg/g of muscimol. In embodiments, the refluxed extract comprises between about 50 and 300 μg/g of muscimol. In embodiments, the refluxed extract comprises between about 50 and 200 μg/g of muscimol. In embodiments, the refluxed extract comprises between about 100 and 200 μg/g of muscimol. In embodiments, the refluxed extract comprises between about 100 and 150 μg/g of muscimol. In embodiments, the refluxed extract comprises between about 100 and 130 μg/g of muscimol. In embodiments, the refluxed extract comprises about 73 μg/g of muscimol. In embodiments, the refluxed extract comprises about 95 μg/g of muscimol. In embodiments, the refluxed extract comprises about 106 μg/g of muscimol. In embodiments, the refluxed extract comprises about 123 μg/g of muscimol. In embodiments, the refluxed extract comprises about 126 μg/g of muscimol.

In embodiments, the refluxed extract comprises between about 1 and 30 μg/g of muscarine. In embodiments, the refluxed extract comprises between about 5 and 20 μg/g of muscarine. In embodiments, the refluxed extract comprises between about 5 and 15 μg/g of muscarine. In embodiments, the refluxed extract comprises between about 7 and 10 μg/g of muscarine. In embodiments, the refluxed extract comprises about 7 μg/g of muscarine. In embodiments, the refluxed extract comprises about 8 μg/g of muscarine. In embodiments, the refluxed extract comprises about 9 μg/g of muscarine. In embodiments, the refluxed extract comprises about 10 μg/g of muscarine.

In embodiments, the refluxed extract comprises between about 1 and 30 μg/g of ibotenic acid. In embodiments, the refluxed extract comprises between about 2 and 20 μg/g of ibotenic acid. In embodiments, the refluxed extract comprises between about 3 and 15 μg/g of ibotenic acid. In embodiments, the refluxed extract comprises between about 4 and 12 μg/g of ibotenic acid. In embodiments, the refluxed extract comprises less than about 1 μg/g of ibotenic acid. In embodiments, the refluxed extract comprises about 0.6 μg/g of ibotenic acid. In embodiments, the refluxed extract comprises about 5 μg/g of ibotenic acid. In embodiments, the refluxed extract comprises about 7 μg/g of ibotenic acid. In embodiments, the refluxed extract comprises about 11 μg/g of ibotenic acid.

In embodiments, the refluxed extract comprises between about 100 and 130 μg/g of muscimol, between about 7 and 10 μg/g of muscarine, and between about 4 and 12 μg/g of ibotenic acid.

e. Basification

In embodiments, the pH of the extract is then increased by addition of a suitable base. Increasing the pH of the extract can confer advantageous properties to the extract, such as ease of processing, or improved stability. In embodiments, the extract is cooled (e.g., to a temperature of below about 40° C.) prior to basification.

In embodiments, the pH of the extract is raised from about 2.6 (the approximate pH of the extract after refluxing) to a pH of about 4 to about 11. For example, in some embodiments, the pH of the extract is raised to a pH of about 4, 4.5, 5, 6, 7, 8, 9, 10, 11, or PH levels in between these values. In embodiments, the pH of the extract is raised to between about 3.5 and 4.5. In embodiments, the pH of the extract is raised to between about 3.9 and 4.1. In embodiments, the pH of the extract is raised to about 4.

In embodiments, the base is a hydroxide base (e.g., NaOH, KOH, Mg(OH)₂, Ca(OH)₂). In embodiments, the base is a carbonate base (e.g., Na₂CO₃, K₂CO₃, MgCO₃, CaCO₃). In embodiments, the base is a bicarbonate base (e.g., NaHCO₃, KHCO₃, NH₄HCO₃). In embodiments, the base is added in solid form (e.g., as pellets, granules, or powder). In other embodiments, the base is added as a solution, such as an aqueous solution (e.g., aqueous NaOH). In embodiments, the base is 1 M aqueous NaOH. The base can alternatively be another known compound capable of raising the pH of the extract.

The addition of a base to an acidic extract will produce a salt. For example, the addition of NaOH to an acidic extract containing HCl will produce a sodium chloride (NaCl) salt. In embodiments, the amount of base that is added to the extract is titrated in order to achieve a desired PH level (such as pH of about 4) while limiting the formation of excess salt (e.g., NaCl) that may produce an undesirable salty taste in the resulting extract.

f. Formulation and Packaging

In embodiments, the extract is formulated into a product for use, such as for ingestion or consumption by a human or other mammal.

In embodiments, the extract is mixed with an excipient, such as distilled water, an alcohol such as ethanol, or a food grade carrier oil, such as MCT oil, coconut oil, or hemp seed oil, and optionally a polyol (e.g., vegetable glycerin) and/or a lecithin (e.g., soy or sunflower lecithin). In embodiments, the extract is mixed with distilled water. In embodiments, a preservative is optionally added (e.g., sodium bisulfate, sodium benzoate, sodium citrate, potassium sorbate, citric acid). In embodiments, sodium benzoate and potassium sorbate are added. In embodiments, the extract is mixed with distilled water, sodium benzoate, and potassium sorbate.

In embodiments, additional agents are added, e.g., one or more flavors (such as corn flavoring), sweeteners (including artificial sweaters), vitamins, active ingredients (e.g., antioxidants or anti-inflammatoires), herbal extracts, essential oils, and/or any one or more of such other agents as described herein. In embodiments, sufficient further excipient is added to produce a desired volume.

For example, suspensions or other aqueous or liquid formulations for human consumption may be prepared in volumes of 5 mL, 10 mL, 25 mL, 30 mL, 50 mL, 100 mL, or such other total volumes as practical, such as for use or sale as a pharmaceutical or OTC preparation, or a wellness-promoting nutraceutical preparation or dietary supplement. Depending on unit dosage volume and total volume, the extract may be packaged in dropper bottles (e.g., 30 mL/1 oz. bottles) or fine mist spray (i.e., oral spray) bottles (e.g., 10 mL bottles). Liquid suspensions also can be used to prepare softgel capsules, ampoules, or other single unit dosage forms, through methods herein disclosed or known to those of skill.

In one specific example, a 110 L jacketed tank fitted with a mechanical overhead stirrer is used to contain and stir the extract. Distilled water is added, and in this example, sodium benzoate and potassium sorbate are added as preservatives. A corn flavoring agent is added to the extract.

In embodiments, the extract is filtered (e.g., using a 0.2-micron filter) after formulation and before packaging. In other embodiments, the extract is not filtered after formulation and before packaging.

### C. AMANITA MUSCARIA EXTRACTS

In some aspects, provided are wellness-promoting extracts of A. muscaria obtained according to disclosed methods.

In embodiments, a provided A. muscaria extract comprises muscimol. Muscimol is one of the main psychoactive components of A. muscaria. Muscimol is known to be an agonist for GABAA receptors (Johnston. Neurochem Res. 2014; 39 (10): 1942-7). When binding to a GABAA receptor, muscimol activates the receptor, causing anxiolytic, anticonvulsant, amnesic, sedative, hypnotic, euphoriant, and muscle relaxant properties. Muscimol may also cause hallucinogenic effects in a subject.

While the toxicokinetics of muscimol remain largely unknown, a fast absorption of muscimol is presumed based on the rapid onset of symptoms after ingestion of Amanita mushrooms (Stříbrný et al. Int J Legal Med. 2012; 126:519-524). Moreover, muscimol is readily excreted into the urine, where it can be detected within an hour after ingestion (Stříbrný et al. Int J Legal Med. 2012; 126:519-524). Preclinical studies in rats investigated the distribution and metabolism of muscimol. Baraldi and colleagues investigated these in the brain and other tissues in the rat. Male Sprague Dawley rats (n=3-5) received either an IV injection of muscimol (3Hmethylene-muscimol, 8 μmol/kg or 12.1 Ci/mmol) or intracerebroventricular injection (1200 nmol/kg), and metabolites from blood and brain were extracted. Intravenous administration of muscimol resulted in an uneven distribution at various brain regions, with the highest muscimol concentration being in the substantia nigra, the colliculi and the hypothalamus. However, only a small amount of the dose injected (approximately 0.02% in a 150 g rat) crossed to the brain (approximately 200 pmol/g tissue). On the other hand, intracerebroventricular injection of muscimol led to a distribution of muscimol that was approximately 2 times higher in the corneum stratum compared to the hypothalamus, hippocampus and cortex. After intravenous injection, muscimol was removed very rapidly from the blood, and blood concentration was below limits of detection within 180 min. Upon disappearance from the blood, there was a paralleled increase in plasma levels of radioactive muscimol metabolites. Furthermore, there were no reports of adverse effects or toxicity in this study, suggesting that an intravenous injection of muscimol (8 μmol/kg) was well-tolerated (Baraldi et al. Neuropharmacology. 1979; 18 (1): 57-62).

In embodiments, the A. muscaria extract comprises between about 0.001% and 1.0% (w/w) of muscimol. In embodiments, the A. muscaria extract comprises between about 0.005% and 0.1% (w/w) of muscimol. In embodiments, the A. muscaria extract comprises between about 0.01% and 0.1% (w/w) of muscimol. In embodiments, the A. muscaria extract comprises between about 0.01% and 0.02% (w/w) of muscimol. In embodiments, the A. muscaria extract comprises about 0.010% (w/w) of muscimol. In embodiments, the A. muscaria extract comprises about 0.011% (w/w) of muscimol. In embodiments, the A. muscaria extract comprises about 0.012% (w/w) of muscimol. In embodiments, the A. muscaria extract comprises about 0.013% (w/w) of muscimol. In embodiments, the A. muscaria extract comprises about 0.014% (w/w) of muscimol. In embodiments, the A. muscaria extract comprises about 0.015% (w/w) of muscimol.

In embodiments, the A. muscaria extract comprises between about 50 and 500 μg/g of muscimol. In embodiments, the A. muscaria extract comprises between about 50 and 400 μg/g of muscimol. In embodiments, the A. muscaria extract comprises between about 50 and 300 μg/g of muscimol. In embodiments, the A. muscaria extract comprises between about 50 and 200 μg/g of muscimol. In embodiments, the A. muscaria extract comprises between about 100 and 200 μg/g of muscimol. In embodiments, the A. muscaria extract comprises between about 100 and 150 μg/g of muscimol. In embodiments, the A. muscaria extract comprises between about 100 and 130 μg/g of muscimol. In embodiments, the A. muscaria extract comprises about 73 μg/g of muscimol. In embodiments, the A. muscaria extract comprises about 95 μg/g of muscimol. In embodiments, the A. muscaria extract comprises about 106 μg/g of muscimol. In embodiments, the A. muscaria extract comprises about 123 μg/g of muscimol. In embodiments, the A. muscaria extract comprises about 126 μg/g of muscimol.

In embodiments, a provided A. muscaria extract comprises muscarine. Muscarine is a further component of A. muscaria that is typically found in smaller quantities than ibotenic acid and muscimol. Muscarine is a nonselective agonist of the muscarinic acetylcholine (ACh) receptors (Broadley et al. Molecules. 2001; 6 (3): 142-193), which may be toxic in concentrations found in certain species, but not typically in those found in A. muscaria.

Muscarine induces short-lived symptoms, indicative of activation of muscarinic receptors, including vomiting, hypotension, nausea, abdominal cramping, diarrhea, lacrimation, hypersalivation, bronchoconstriction, chills, tremor, bronchial secretions, sweating, gastric acid secretion, miosis, blurred vision, polyuria, diaphoresis, rhinorrhea, headache, anxiety and bradycardia (Lurie et al. Clin Toxicol (Phila). 2009; 562-565). Despite such symptoms, it is important to consider that in terms of muscarinic syndrome from ingestion of A. muscaria, one would have to ingest enormous quantities of A. muscaria before a muscarinic effect is elicited.

Muscarine mimics the action of acetylcholine (ACh) on muscarinic-type ACh receptors. In general, quaternary ammonium compounds such as muscarine are poorly absorbed after oral exposure. Once absorbed, muscarine is quickly distributed throughout the body, and clinical signs can develop in humans within 30 min to 2 hours. Muscarine is not metabolized by acetylcholinesterase like the neurotransmitter ACh. Instead, muscarine leaves the blood via renal clearance and exits the body in urine (Bartholow, R. A practical treatise on materia medica and therapeutics. D. Appleton & Company, 1876). While detailed pharmacokinetic studies on muscarine are scarce, recently, Sai Latha et al investigated the pharmacokinetics and biodistribution of a synthetic radiolabelled muscarine (Sai Latha et al. Sci Rep. 2020; 10 (1): 13669). In this study, radiolabelled muscarine chloride (1.5 mCi 99mTcO4-) was injected through the dorsal ear vein of normal, healthy female New Zealand rabbits (n=3). Blood was withdrawn at periodic intervals and radioactivity measured using gamma scintigraphy. Approximately 42% of the radiolabelled muscarine reached the blood within 5 min after administration. A significant amount of the toxin was rapidly concentrated in the thorax and head, demonstrating early muscarinic responses such as miosis and salivation. Muscarine was also detected in the spleen following a decline of blood level by 87.89% after 1 hr of ingestion. Muscarine was detected in the bladder, suggesting renal clearance. After 24 hours, a large proportion of muscarine was accumulated in the liver, which provides a potential explanation for the hepatoxicity that was observed in the study in mice.

In embodiments, the A. muscaria extract comprises between about 0.0001% and 0.0010% (w/w) of muscarine. In embodiments, the A. muscaria extract comprises between about 0.0002% and 0.0010% (w/w) of muscarine. In embodiments, the A. muscaria extract comprises between about 0.0003% and 0.0010% (w/w) of muscarine. In embodiments, the A. muscaria extract comprises between about 0.0004% and 0.0010% (w/w) of muscarine. In embodiments, the A. muscaria extract comprises between about 0.0005% and 0.0010% (w/w) of muscarine. In embodiments, the A. muscaria extract comprises between about 0.0006% and 0.0010% (w/w) of muscarine. In embodiments, the A. muscaria extract comprises between about 0.0001% and 0.0010% (w/w) of muscarine. In embodiments, the A. muscaria extract comprises about 0.0006% (w/w) of muscarine. In embodiments, the A. muscaria extract comprises about 0.0007% (w/w) of muscarine. In embodiments, the A. muscaria extract comprises about 0.0008% (w/w) of muscarine. In embodiments, the A. muscaria extract comprises about 0.0009% (w/w) of muscarine. In embodiments, the A. muscaria extract comprises about 0.0010% (w/w) of muscarine.

In embodiments, the A. muscaria extract comprises between about 1 and 30 μg/g of muscarine. In embodiments, the A. muscaria extract comprises between about 5 and 20 μg/g of muscarine. In embodiments, the A. muscaria extract comprises between about 5 and 15 μg/g of muscarine. In embodiments, the A. muscaria extract comprises between about 7 and 10 μg/g of muscarine. In embodiments, the A. muscaria extract comprises about 7 μg/g of muscarine. In embodiments, the A. muscaria extract comprises about 8 μg/g of muscarine. In embodiments, the A. muscaria extract comprises about 9 μg/g of muscimol. In embodiments, the A. muscaria extract comprises about 10 μg/g of muscarine.

In embodiments, a provided A. muscaria extract comprises ibotenic acid. Ibotenic acid is a conformationally restricted analogue of the neurotransmitter glutamate that acts as a non-selective glutamate receptor agonist. Ibotenic acid also acts as a neurotoxin and has been employed as a “brain-lesioning agent” through cranial injections in scientific research. In typical samples, A. muscaria contains more ibotenic acid than muscimol. At least some of the ibotenic acid in A. muscaria is converted by decarboxylation to muscimol in the acid environment of the stomach. Ibotenic acid therefore can serve as a prodrug to muscimol. However, if too much ibotenic acid is ingested, it can cause stomach irritation, nausea, diarrhea, sweating and salivation, lethargy and drowsiness, ataxia, and other somatic symptoms, as well as psychological symptoms such as confusion, euphoria, visual and auditory hallucinations, sensations of floating, distortions of space and time, and retrograde amnesia (Moss et al. Clin Toxicol (Phila). 2019; 57 (2): 99-103).

Ibotenic acid is the precursor of muscimol. It is metabolized by decarboxylation in the stomach, liver, and brain to equal amounts of muscimol (Nielsen et al. J Neurochem. 1985; 45:725-731). While the toxicokinetics of ibotenic acid remain largely unknown, a fast absorption of ibotenic acid is presumed after ingestion of Amanita mushrooms due to the rapid appearance of related symptoms (Stříbrný et al. Int J Legal Med, 2012; 126:519-524). Similar to muscimol, ibotenic acid is also readily excreted into the urine, where it can be detected within an hour after ingestion (Stříbrný et al. Int J Legal Med, 2012; 126:519-524).

In embodiments, the A. muscaria extract comprises between about 0.0001% and 0.0010% (w/w) of ibotenic acid. In embodiments, the A. muscaria extract comprises less than about 0.0001% (w/w) of ibotenic acid. In embodiments, the A. muscaria extract comprises between about 0.0002% and 0.0010% (w/w) of ibotenic acid. In embodiments, the A. muscaria extract comprises between about 0.0003% and 0.0010% (w/w) of ibotenic acid. In embodiments, the A. muscaria extract comprises between about 0.0004% and 0.0010% (w/w) of ibotenic acid. In embodiments, the A. muscaria extract comprises between about 0.0005% and 0.0010% (w/w) of ibotenic acid. In embodiments, the A. muscaria extract comprises between about 0.0006% and 0.0010% (w/w) of ibotenic acid. In embodiments, the A. muscaria extract comprises between about 0.0001% and 0.0010% (w/w) of ibotenic acid. In embodiments, the A. muscaria extract comprises about 0.0001% (w/w) of ibotenic acid. In embodiments, the A. muscaria extract comprises about 0.0002% (w/w) of ibotenic acid. In embodiments, the A. muscaria extract comprises about 0.0003% (w/w) of ibotenic acid. In embodiments, the A. muscaria extract comprises about 0.0004% (w/w) of ibotenic acid. In embodiments, the A. muscaria extract comprises about 0.005% (w/w) of ibotenic acid.

In embodiments, the A. muscaria extract comprises between about 1 and 30 μg/g of ibotenic acid. In embodiments, the A. muscaria extract comprises between about 2 and 20 μg/g of ibotenic acid. In embodiments, the A. muscaria extract comprises between about 3 and 15 μg/g of ibotenic acid. In embodiments, the A. muscaria extract comprises between about 4 and 12 μg/g of ibotenic acid. In embodiments, the A. muscaria extract comprises about 0.6 μg/g of ibotenic acid. In embodiments, the A. muscaria extract comprises about 1 μg/g of ibotenic acid. In embodiments, the A. muscaria extract comprises about 2 μg/g of muscimol. In embodiments, the A. muscaria extract comprises about 3 μg/g of ibotenic acid. In embodiments, the A. muscaria extract comprises about 4 μg/g of ibotenic acid. In embodiments, the A. muscaria extract comprises about 5 μg/g of ibotenic acid. In embodiments, the A. muscaria extract comprises about 6 μg/g of ibotenic acid. In embodiments, the A. muscaria extract comprises about 7 μg/g of ibotenic acid. In embodiments, the A. muscaria extract comprises about 8 μg/g of ibotenic acid. In embodiments, the A. muscaria extract comprises about 9 μg/g of ibotenic acid. In embodiments, the A. muscaria extract comprises about 10 μg/g of ibotenic acid.

In embodiments, the A. muscaria extract is substantially or essentially devoid of stizolobinic acid. In embodiments, the A. muscaria extract comprises less than 40 μg/g of stizolobinic acid. In embodiments, the A. muscaria extract is substantially or essentially devoid of heavy metals. In embodiments, the A. muscaria extract is substantially or essentially devoid of one or more of cadmium, arsenic, lead, and mercury. In embodiments, the A. muscaria extract comprises less than 0.09 ppm of cadmium, 0.03 ppm of arsenic, 0.09 ppm of lead and 0.02 ppm of mercury. In embodiments, the A. muscaria extract comprises no more than 40 μg/g stizolobinic acid and no more than 0.09 ppm of cadmium, 0.03 ppm of arsenic, 0.09 ppm of lead and 0.02 ppm of mercury.

In embodiments, the A. muscaria extract comprises less than or no more than the limits for respective pesticides in USP General Chapter 561, “Articles of Botanical Origin” (USP 561). In embodiments, the extract is also in compliance with EPA (40 C.F.R. § 180) and FDA action levels (21 C.F.R. §§ 109, 509). In embodiments, the extract is manufactured in compliance with GLP or GMP requirements.

One of skill may use known methods to determine the presence of contaminants, one such method being HPLC-MS/MS. In embodiments, HPLC-MS/MS may be used to determine the presence and concentration of other target compounds in the A. muscaria extract. In embodiments, HPLC-MS/MS is used to determine the concentration of muscimol, ibotenic acid, and/or muscarine.

In embodiments, the A. muscaria extract comprises muscimol and muscarine in a weight ratio of at least 1:1. In embodiments, the muscimol to muscarine ratio is from about 1:1 to about 100:1. In embodiments, the muscimol to muscarine ratio is about 1:1, 5:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, and 100:1.

In embodiments, the weight ratio of muscimol to ibotenic acid in the A. muscaria extract is between about 10:1 and 1000:1. In embodiments, the weight ratio of muscimol to ibotenic acid in the A. muscaria extract is between about 10:1 and 500:1. In embodiments, the weight ratio of muscimol to ibotenic acid in the A. muscaria extract is between about 10:1 and 400:1. In embodiments, the weight ratio of muscimol to ibotenic acid in the A. muscaria extract is between about 10:1 and 300:1. In embodiments, the weight ratio of muscimol to ibotenic acid in the A. muscaria extract is between about 10:1 and 200:1. In embodiments, the weight ratio of muscimol to ibotenic acid in the A. muscaria extract is between about 10:1 and 100:1. In embodiments, the weight ratio of muscimol to ibotenic acid in the A. muscaria extract is between about 10:1 and 50:1. In embodiments, the weight ratio of muscimol to ibotenic acid in the A. muscaria extract is between about 10:1 and 40:1. In embodiments, the weight ratio of muscimol to ibotenic acid in the A. muscaria extract is between about 10:1 and 30:1. In embodiments, the weight ratio of muscimol to ibotenic acid in the A. muscaria extract is between about 10:1 and 20:1. In embodiments, the weight ratio of muscimol to ibotenic acid in the A. muscaria extract is between about 10:1 and 15:1.

In embodiments, the A. muscaria extract comprises muscimol and ibotenic acid in a weight ratio of at least 3:1. In embodiments, the muscimol to ibotenic acid ratio is from about 3:1 to about 150:1. In embodiments, the muscimol to ibotenic acid ratio is about 10:1, 12.5:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 105:1, 110:1, 115:1, 120:1, 125:1, 130:1, 135:1, 140:1, 145:1, 150:1, 155:1, 160:1, 165:1, 170:1, 175:1, 180:1, 185:1, 190:1, 195:1, or 200:1.

In embodiments, the A. muscaria extract comprises muscimol and ibotenic acid in a weight ratio of about 1000:1 to 3000:1, 1500:1 to 2500:1, or 1750:1 to 2250:1. In embodiments, the A. muscaria extract comprises muscimol and ibotenic acid in a weight ratio of at least 1000:1, at least 1100:1, at least 1200:1, at least 1300:1, at least 1400:1, at least 1500:1, at least 1600:1, at least 1700:1, at least 1800:1, at least 1900:1, at least 2000:1, at least 2100:1, at least 2200:1, at least 2300:1, at least 2400:1, at least 2500:1, at least 2600:1, at least 2700:1, at least 2800:1, at least 2900:1, or at least 3000:1. In embodiments, the A. muscaria extract comprises muscimol and ibotenic acid in a weight ratio of about 1500:1, about 1510:1, about 1520:1, about 1530:1, about 1540:1, about 1550:1, about 1560:1, about 1570:1, about 1580:1, about 1590:1, about 1600:1, about 1610:1, about 1620:1, about 1630:1, about 1640:1, about 1650:1, about 1660:1, about 1670:1, about 1680:1, about 1690:1, about 1700:1, about 1710:1, about 1720:1, about 1730:1, about 1740:1, about 1750:1, about 1760:1, about 1770:1, about 1780:1, about 1790:1, 1800:1, about 1810:1, about 1820:1, about 1830:1, about 1840:1, about 1850:1, about 1860:1, about 1870:1, about 1880:1, about 1890:1, about 1900:1, about 1910:1, about 1920:1, about 1930:1, about 1940:1, about 1950:1, about 1960:1, about 1970:1, about 1980:1, about 1990:1, or about 2000:1, about 2010:1, about 2020:1, about 2030:1, about 2040:1, about 2050:1, about 2060:1, about 2070:1, about 2080:1, about 2090:1, or about 3000:1.

In embodiments, the A. muscaria extract comprises trehalose. In embodiments, the concentration of trehalose in the A. muscaria extract is between about 0.01 M and 0.20 M. In embodiments, the concentration of trehalose is between 0.01 M and 0.05 M, 0.06 M and 0.10 M, 0.11 M and 0.15 M, or 0.16 M and 0.20 M. In embodiments, the concentration of trehalose is about 0.10 M. In embodiments, the concentration of trehalose is about 0.90 M, or about 0.91 M.

In embodiments, the A. muscaria extract comprises between about 0.001% w/w or w/v muscazone, and about 0.01% w/w or w/v muscazone.

In embodiments, the properties of a provided A. muscaria extract meet the testing specifications shown in TABLES 1-6.

TABLE 1
Testing Specifications for Exemplary A. muscaria Extract

| Parameter | Specification | Method |
|---|---|---|
| Color | Brown | Organoleptic |
| Taste | Similar to meat gravy | Organoleptic |
| Smell | Forest mushroom scent | Organoleptic |
| Muscimol | <30000 μg/g | HPLC-MS/MS |
| Muscarine | <550 μg/g | HPLC-MS/MS |
| Ibotenic acid | <20 μg/g | HPLC-MS/MS |
| Stizolobinic acid | <40 μg/g | HPLC-MS/MS |
| Arsenic | <0.03 ppm | HPLC-MS/MS |
| Cadmium | <0.09 ppm | HPLC-MS/MS |
| Mercury | <0.02 ppm | HPLC-MS/MS |
| Lead | <0.09 ppm | HPLC-MS/MS |
| Pesticides | Conforms | Conforms |

TABLE 2
Concentrations of Amanita Alkaloids in Exemplary
A. muscaria Extract

| | LOD* | LOQ** | Result | |
|---|---|---|---|---|
| Analyte | (%) | (%) | (μg/g) | % |
| Muscimol | 8.81 × 10⁻⁶ | 5.21 × 10⁻⁵ | 106 | 0.0103 |
| Muscarine | 3.65 × 10⁻⁶ | 1.3 x 10⁻⁵ | 7.77 | 0.000755 |
| Ibotenic acid | 1.13 × 10⁻⁵ | 5.21 x 10⁻⁵ | 4.61 | 0.000448 |

Total Alkaloids (Total Amanita Analytes) = 0.0115% (3.551 mg per 30 mL)
*LOD = Level of Detection;
**LOQ = Level of Quantification

TABLE 3
Concentrations of Alkaloids in Exemplary A. muscaria
Extract Determined by HPLC-MS/MS

| Compound | Result (μg/g) |
|---|---|
| Ibotenic Acid | 0.674 |
| Muscarine | 9.56 |
| Muscimol | 73.4 |

TABLE 4
Concentrations of Alkaloids in Exemplary A. muscaria
Extract Determined by HPLC-MS/MS

| Compound | Result (μg/g) |
|---|---|
| Ibotenic Acid | 0.612 |
| Muscarine | 7.98 |
| Muscimol | 95.2 |

TABLE 5
Concentrations of Alkaloids in Exemplary A. muscaria
Extract Determined by HPLC-MS/MS

| Compound | Result (μg/g) |
|---|---|
| Ibotenic Acid | 7.54 |
| Muscarine | 8.19 |
| Muscimol | 123 |

TABLE 6
Concentrations of Alkaloids in Exemplary A. muscaria
Extract Determined by HPLC-MS/MS

| Compound | Result (μg/g) |
|---|---|
| Ibotenic Acid | 11.5 |
| Muscarine | 8.05 |
| Muscimol | 126 |

In some aspects, provided herein are A. muscaria compositions of specific potency and purity. In embodiments, potency is equivalent to concentration. In embodiments, potency can be determined by w/w %, e.g., by dividing the weight of a compound to be assessed with the total weight of an extract. Potency and purity may be determined according to methods known to one of skill in the art.

In embodiments, the potency of muscimol in a disclosed A. muscaria composition, such as an extract, is at least 0.25%, 0.5%, 0.75%, 1%, 1.25%, 1.5%, 1.6%, 1.65%, 1.75%, 2.0%, 2.5%, 2.75%, or 3%. In embodiments, the potency of muscimol in a disclosed A. muscaria composition is about 0.25% to 5%, 0.5% to 4%, 0.75% to 3%, 1% to 2%, 1.25% to 1.75%, 1.5% to 1.7%. In embodiments, a disclosed A. muscaria compositions, such as an extract, has a muscimol potency of about 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0%.

In embodiments, the potency of muscarine in a disclosed A. muscaria composition, such as an extract, is less than 0.02%, 0.03%, 0.04%, 0.05%, 0.06%, 0.07%, 0.08%, 0.09% or 1%. In embodiments, the potency of muscarine in a disclosed A. muscaria composition is about 0.01% to 0.3%, 0.025% to 0.2%, 0.03% to 0.175%, or 0.04% to 0.15%. In embodiments, a disclosed A. muscaria compositions, such as an extract, has a muscarine potency of about 0.03%, 0.04%, 0.05%, 0.06%, 0.07%, 0.08%, 0.09%, 0.1%, 0.15%, 0.2%, 0.25%, 0.3%, 0.35%, or 0.4%

In embodiments, the A. muscaria extract comprises ibotenic acid having a potency of less than 0.025%, 0.02%, 0.015%, 0.01%, 0.009%, 0.008%, 0.007%, 0.006%, 0.005%, 0.004%, 0.0035%, 0.003%, 0.0025%, 0.002%, 0.0015%, or 0.001%.

In embodiments, the A. muscaria extract has a muscimol purity of at least 90%, such as about 90%, 91%, 92%, 93%, 94%, 95%, 96%, 97%, 98%, 99%, and greater than 99%. In embodiments, the purity of a compound is determined by dividing the weight of the compound by the total weight of the compounds against which its purity is measured (i.e., as a w/w %).

In embodiments, the A. muscaria extract, which may optionally be further concentrated, is standardized. In embodiments, the A. muscaria extract is standardized to a muscimol purity of 90% or greater. A “standardized” extract refers to an extract comprising a specified quantity of a standardized ingredient, which may be a bioactive compound such as muscimol. Thus, In embodiments, an amount of the bioactive compound, such as an amount of muscimol, is standardized to a particular concentration (e.g., w/w or w/v % of the extract). In embodiments, an A. muscaria extract will be standardized so as to contain by weight percent an amount of muscimol (i.e., mg muscimol per mg or mL of extract, depending on whether such extract is a dry powder or a liquid) of between 0.5% and 5.0% w/w or w/v muscimol, wherein the range is inclusive. In embodiments, the A. muscaria extract will contain by weight percent an amount of muscarine (i.e., mg muscarine per mg extract) of at least 0.05% w/w or w/v muscarine. Other standardizations of w/w or w/v muscimol will include, for example, amounts of between 5.0% and 10.0% or greater than 10% muscimol and also including amounts lower than those explicitly listed above.

Standardization may be accomplished by methods such as measuring a concentration of compound in an extract to be standardized, determining a desired concentration of the compound when standardized, determining an amount of excipient necessary to obtain the desired (standardized) concentration, and then adding the amount of excipient necessary to obtain the desired (standardized) concentration, resulting in a standardized extract. An excipient may be a dry or liquid excipient, to create a dry powder or liquid standardized extract.

The concentration of the standardized compound in the standardized extract may be measured after adding one or more portions of excipient or after the standardized extract is prepared, to confirm the standardization method and for quality control.

In embodiments, the A. muscaria extract is further concentrated so the bioactive compounds (including, and in particular muscimol) are increased in total concentration from an initial extract, such as an increase in w/w % (for a powder extract) or w/v % (for a liquid extract), in an amount such as by at least 5%, at least 10%, at least 15%, at least 20%, at least 25%, at least 30%, at least 40%, at least 45%, at least 50%, at least 60%, at least 70%, at least 80%, at least 90%, at least 100% (2×), at least 125%, at least 150%, at least 175%, at least 200% (3×), at least 250%, at least 300% (4×), at least 400% (5×), at least 500% (6×), at least 600% (7×), at least 700% (8×), at least 800% (9×), at least 900% (10×), and in amounts of 1,000% or more.

In some embodiments having a further concentrated extract, the bioactive compounds in the A. muscaria extract may be increased by a like amount. For example, where a pharmaceutical composition includes a 10× concentrated A. muscaria extract, it will contain bioactive A. muscaria compounds including muscimol, muscarine, and ibotenic acid, and will as an example contain by weight/weight or weight/volume percent an amount of muscimol of 5% or less, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, or at least 30% w/w muscimol.

Exemplary methods of concentrating an extract include, e.g., evaporating a portion or an entirety of a solvent to create a volume of concentrated slurry at a desired concentration.

### D. APPLICATIONS AND USES

Disclosed wellness-promoting extracts have applications for the improvement of human health, including to reduce pain and treat pain disorders, to reduce and treat inflammation and inflammatory disorders, to benefit immunity and reduce or treat symptoms of immune disorders, including autoimmune diseases and disorders, and for the general improvement of physical health and wellness including relaxation and improvement in sleep, as illustrative and non-limiting examples.

In some aspects are provided methods of modulating neurotransmission comprising administering the disclosed extract to a subject, thereby modulating neurotransmission in said subject. In embodiments, the neurotransmission is one or more of gabaminergic neurotransmission, glutaminergic neurotransmission, and cholinergic neurotransmission. In some aspects are provided methods of treating a health condition, comprising administering to a patient an effective amount of the disclosed extract, compound, or pharmaceutical composition. In embodiments, the health condition is a mental health disorder. In embodiments, the mental health disorder is selected from depression, dysthymia, an anxiety and phobia disorders, generalized anxiety disorder, social anxiety disorder, panic disorder, post-traumatic stress disorder, an adjustment disorders, a feeding and eating disorders, binge eating disorder, bulimia, and anorexia nervosa, other binge behaviors, body dysmorphic syndromes, alcoholism, tobacco abuse, drug abuse or dependence disorders, disruptive behavior disorders, impulse control disorders, gaming disorders, gambling disorders, memory loss, dementia of aging, attention deficit hyperactivity disorder, personality disorders, antisocial personality disorder, avoidant personality disorder, borderline personality disorder, histrionic personality disorder, narcissistic personality disorder, obsessive compulsive disorder, paranoid personality disorder, schizoid personality disorder, schizotypal personality disorders, attachment disorders, autism, and dissociative disorders. In embodiments, the mental health disorder is an anxiety disorder. In embodiments, the anxiety disorder is any of acute stress disorder, anxiety due to a medical condition, generalized anxiety disorder, panic disorder, panic attack, a phobia, post traumatic stress disorder (PTSD), separation anxiety disorder, social anxiety disorder, substance-induced anxiety disorder, and selective mutism. In embodiments, the mental health disorder is a substance use disorder. In embodiments, the substance use disorder is any of alcohol use disorder, cannabis use disorder, hallucinogen use disorder, inhalant use disorder, opioid use disorder, sedative use disorder, stimulant use disorder, tobacco use disorder, and nicotine use disorder. In embodiments, the mental health disorder is a behavioral addiction. In embodiments, the behavioral addiction is selected from gambling disorder, gaming disorder, sexual addiction, compulsive buying disorder, and technology addiction. In embodiments, the health condition is a sleep disorder. In embodiments, the sleep disorder is any of an insomnia, a hypersomnia, a parasomnia, and a disorder of sleep-wake schedule.

In embodiments, the health disorder is a physical health disorder. In embodiments, the physical health disorder is a pain disorder. In embodiments, the pain disorder is any of arthritis, allodynia, atypical trigeminal neuralgia, trigeminal neuralgia, somatoform disorder, hypoesthesia, hyperalgesia, neuralgia, heuritis, neurogenic pain, analgesia, anesthesia dolorosa, causalgia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis, or pain associated with cancer. In embodiments, the physical health disorder is a disorder that causes acute inflammation, or that exhibits chronic inflammation as a symptom.

In embodiments, the physical health disorder is an autoimmune disorder. In embodiments, the autoimmune disorder is any of acute disseminated encephalomyelitis (ADEM), Addison disease, allergy or hypersensitivity, amyotrophic lateral sclerosis, antiphospholipid antibody syndrome (APS), arthritis, autoimmune hemolysis Anemia, autoimmune hepatitis, autoimmune inner ear disease, autoimmune pancreatitis, bullous pemphigoid, celiac disease, Chagas disease, chronic obstructive pulmonary disease (COPD), type 1 diabetes (T1D), endometriosis, fibromyalgia, goodpasture's syndrome, Graves' disease, Guillain-Barre syndrome (GBS), Hashimoto's thyroiditis, suppurative spondylitis, idiopathic thrombocytopeni purpura, inflammatory bowel disease, interstitial cystitis, lupus, including discoid lupus erythematosus, drug-induced lupus lupus erythematosus, lupus nephritis, neonatal lupus, subacute cutaneous lupus erythematosus, and systemic lupus erythematosus; morphea, multiple hard Keratosis (MS), myasthenia gravis, myopathy, narcolepsy, neuromuscular angina, pemphigus vulgaris, pernicious anemia, primary biliary cirrhosis, recurrent diffuse encephalomyelitis, including polyphasic diffuse encephalomyelitis, rheumatic fever, schizophrenia, scleroderma, Sjogren's syndrome, tendonitis, vasculitis, and vitiligo. In embodiments, the autoimmune disorder is a systemic autoimmune disorder, including systemic lupus erythematosus (SLE), Sjogren's syndrome, scleroderma, rheumatoid arthritis, and polymyositis. In embodiments, the autoimmune disorder is a local autoimmune disorder, including those of the endocrine system, including type 1 diabetes, Hashimoto's thyroiditis, and Addison's disease; the cutaneous, including pemphigus vulgaris; the blood, including autoimmune hemolytic anemia; and the nervous system, including multiple sclerosis.

In some aspects are provided methods of using the disclosed extract to improve health and wellness, comprising administering an effective amount of the extract, compound, or composition to a subject. In embodiments, the improvement in health and wellness is a reduction in stress. In embodiments, the improvement in health and wellness is an easing of muscular tension. In embodiments, the improvement to health and wellness is a promotion of restorative sleep. In embodiments, the improvement to health and wellness is any of a soothing of the body, a calming of the mind, and a reduction in physical distress. In embodiments, the improvement to health and wellness includes any one or more of a reduction in feelings of nervousness, “jitters,” nervous tension, or anxiety; a reduction in feelings of malaise, unhappiness, existential angst, ennui, and general discontent; and an increase in feelings of wellbeing, wellness, relaxation, contentment, happiness, openness to experience, and life satisfaction. In some aspects are provided methods of using the disclosed extract, compound, or composition to induce euphoria, comprising administering an effective amount of the extract, compound, or composition to an individual.

In accordance with one embodiment of the invention, the Amanita muscaria extract may be prepared for ingestion in the form of a liquid solution, liquid suspension, tincture, beverage concentrate, or beverage, for example, for the purposes described above. In accordance with another embodiment of the invention, the Amanita muscaria extract may be prepared for ingestion in the form of a tablet, a capsule, a softgel, and a gelcap, for the purposes described above, In accordance with another embodiment of the invention, the Amanita muscaria extract may be prepared for topical administration in the form of a cream, an ointment, a gel, a foam, and a liquid composition for transdermal application to alleviate pain, itching, and inflammation, as well as to moisturize, rejuvenate, and provide an immune boost to skin and nearby tissue, for example.

Exemplary embodiments include edible formulations, topical formulations, and vaporizer formulations.

a. Compositions

In some aspects, provided herein are compositions comprising A. muscaria extracts, A. muscaria compounds, or analogs thereof. In embodiments, the compositions are nutraceutical compositions. In embodiments, the compositions are pharmaceutical compositions. Pharmaceutical compositions are compositions that include the compounds together in an amount (for example, in a unit dosage form) with a pharmaceutically acceptable carrier, diluent, or excipient. It should be understood that some embodiments do not have a single carrier, diluent, or excipient alone, but include multiple carriers, diluents, and/or excipients.

Compositions can be prepared by standard pharmaceutical formulation techniques such as disclosed in Remington: The Science and Practice of Pharmacy (2005) 21th ed., Mack Publishing Co., Easton, Pa.; The Merck Index (1996) 12th ed., Merck Publishing Group, Whitehouse, N.J.; Pharm. Principles of Solid Dosage Forms (1993), Technomic Publishing Co., Inc., Lancaster, Pa.; and Ansel and Stoklosa, Pharm. Calculations (2001) 11th ed., Lippincott Williams & Wilkins, Baltimore, Md.; and Poznansky et al. Drug Delivery Systems (1980), R. L. Juliano, ed., Oxford, N.Y., pp. 253-315.

Although disclosed compositions may be referred to as “pharmaceutical” compositions or for “pharmaceutical” purpose or preparation, it will be appreciated that the term simply means that a composition is contemplated or shown to possess therapeutic or beneficial effects when administered for its intended purpose to a mammal, such as a human. It therefore will be understood that the disclosed compositions are useful regardless of the regulatory regime under which they are ultimately sold (e.g., as prescription pharmaceutical drug products or non-prescription over-the-counter (OTC) drug products, or as nutritional supplements or “nutraceuticals”), and also if not sold under a specific regulatory regime at all.

“Pharmaceutically acceptable” as used in connection with one or more ingredients means that the ingredients are generally safe and, within the scope of sound medical judgment, suitable for use in contact with the cells of humans and other animals without undue toxicity, irritation, allergic response, or complication, and commensurate with a reasonable risk/benefit ratio.

In making the disclosed compositions the active ingredients are often mixed with an excipient, diluted by an excipient, or enclosed within such a carrier which can be in the form of a capsule, sachet, paper, or other container. When the excipient serves as a diluent, it can be a solid, semi-solid, or liquid material, which acts as a vehicle, carrier, or medium for the active ingredient. Thus, the compositions can be in the form of tablets, pills, lozenges, sachets, cachets, elixirs, suspensions, emulsions, solutions, syrups, aerosols (as a solid or in a liquid medium), ointments, soft or hard gelatin capsules, suppositories, sterile injectable solutions, and sterile packaged powders. Different embodiments include immediate, delayed, extended, and controlled release forms. Many other variations are possible and known to those skilled in the art.

Examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. Formulations can additionally include: lubricating agents such as talc, magnesium stearate, and mineral oil; wetting agents; emulsifying and suspending agents; preserving agents such as methyl- and propyl-hydroxybenzoates; sweetening agents; and flavoring agents. Compositions can be formulated so as to provide quick, sustained or delayed release of compounds in disclosed extracts after administration by employing procedures known in the art.

In preparing a formulation, it may be necessary to mill an active compound to provide the appropriate particle size prior to combining with the other ingredients. If an active compound is substantially insoluble, it may be milled to a particle size of less than 200 mesh. If an active compound is substantially water soluble, the particle size is normally adjusted by milling to provide a substantially uniform distribution in the formulation, e.g., about 40 mesh.

Pharmaceutical compositions can be formulated into any suitable dosage form, including aqueous or oil-based liquid suspensions or solutions, including tinctures; solid dosage forms, including oral solid dosage forms (e.g., tablets and capsules), sublingual or buccal tablets, confectionary products, beverage concentrates, vaporizer formulations, injectable solutions, topical formulations, transdermal formulations, controlled release formulations, fast melt formulations, delayed-release formulations, immediate-release formulations, modified release formulations, extended-release formulations, pulsatile release formulations, multi particulate formulations, and mixed immediate release and controlled release formulations. Generally speaking, one will desire to administer an amount of the compounds or extracts that is effective to achieve a plasma level commensurate with the concentrations found to be effective in vivo for a period of time effective to elicit the desired therapeutic effect(s).

In embodiments, disclosed extracts are formulated in a unit dosage form. The term “unit dosage form” refers to a physically discrete unit suited as unitary dosages for the subject to be treated, each unit containing a predetermined quantity of active material calculated to produce the desired therapeutic effect(s), in association with a suitable pharmaceutical carrier, diluent, or excipient. Unit dosage forms are often used for ease of administration and uniformity of dosage. Unit dosage forms can contain a single or individual dose or unit, a sub-dose, or an appropriate fraction thereof (e.g., one half a “full” dose), of the pharmaceutical composition administered. Unit dosage forms include capsules, troches, cachets, lozenges, tablets, ampules and vials, which may include a composition in a freeze-dried or lyophilized state; a sterile liquid carrier, for example, can be added prior to administration or delivery in vivo. Unit dosage forms also include ampules and vials with liquid compositions disposed therein. Unit dosage forms further include compounds for transdermal administration, such as “patches” that contact the epidermis of a subject for an extended or brief period of time.

b. Edible Formulations

In embodiments, the disclosed A. muscaria extracts or compositions are provided in an edible formulation for oral administration. Edible formulations include oral solid dosage forms and oral liquid dosage forms.

Oral solid dosage forms include lozenges, troches, oral thin films, softgels, tablets, capsules, caplets, powders, pellets, multiparticulates, beads, spheres, confectionery products (e.g., gummies and infused chocolates), and/or any combinations thereof. Oral solid dosage forms may be formulated as immediate release, controlled release, sustained release, extended release, or modified release formulations. In embodiments, an oral solid dosage form is in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, or granules. In embodiments, an oral solid dosage form is in the form of a powder. In embodiments, the powder may be utilized as, e.g., a beverage concentrate, as a functional food, as a nutritional supplement, and as a nutraceutical powder. Additionally, formulations may be administered as a single capsule or in multiple capsule dosage form. In embodiments, the formulation is administered in 2, 3, 4, or more capsules or tablets.

Oral liquid dosage forms include tinctures, drops, emulsions, syrups, elixirs, suspensions, concentrates, and solutions, and the like. Oral liquid dosage forms may be formulated with any pharmaceutically acceptable excipient known to those of skill for the preparation of liquid dosage forms, and with solvents, diluents, carriers, excipients, and the like chosen as appropriate to the solubility and other properties of the disclosed A. muscaria compositions, and other ingredients. Solvents may be, for example, water, glycerin, simple syrup, alcohol, medium chain triglycerides (MCT), and combinations thereof.

Disclosed edible formulations may contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof. Disclosed edible formulations also can comprise one or more pharmaceutically acceptable additives such as a compatible carrier, complexing agent, ionic dispersion modulator, disintegrating agent, surfactant, lubricant, colorant, moistening agent, plasticizer, stabilizer, penetration enhancer, wetting agent, anti-foaming agent, alone or in combination, as well as supplementary active compound(s).

Supplementary active compounds include preservatives, antioxidants, antimicrobial agents including biocides and biostats such as antibacterial, antiviral and antifungal agents. Preservatives can be used to inhibit microbial growth or increase stability of the active ingredient thereby prolonging the shelf life of the formulation. Suitable preservatives are known in the art and include EDTA, EGTA, benzalkonium chloride or benzoic acid or benzoates, such as sodium benzoate. Antioxidants include vitamin A, vitamin C (ascorbic acid), vitamin E, tocopherols, other vitamins or provitamins, and compounds such as alpha lipoic acid.

Suitable carriers for use in edible formulations include acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose (HPMC), hydroxypropylmethylcellulose acetate stearate (HPMCAS), sucrose, microcrystalline cellulose, lactose, and mannitol.

Suitable filling agents for use in edible formulations include lactose, calcium carbonate, calcium phosphate, dibasic calcium phosphate, calcium sulfate, microcrystalline cellulose, cellulose powder, dextrose, dextrates, dextrose, dextran, starches, pregelatinized starch, HPMC, HPMCAS, hydroxypropylmethylcellulose phthalate, sucrose, xylitol, lactitol, mannitol, sorbitol, sodium chloride, and PEG.

Suitable disintegrants for use in edible formulations include a starch, e.g., a natural starch such as corn starch or potato starch, a pregelatinized starch, or sodium starch glycolate; a cellulose such as a wood product, microcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay; a gum such as agar, guar, locust bean, Karaya, pectin, or tragacanth; sodium starch glycolate; bentonite; a natural sponge; a surfactant; a resin such as a cation-exchange resin; citrus pulp; and sodium lauryl sulfate.

Examples of dispersing agents suitable for the edible formulations include hydrophilic polymers, electrolytes, Tween® 60 or 80, polyethylene glycol (PEG), polyvinylpyrrolidone (PVP), carbohydrate-based dispersing agents, noncrystalline cellulose, magnesium aluminum silicate, triethanolamine, polyvinyl alcohol (PVA), polyvinylpyrrolidone/vinyl acetate copolymer, poloxamers, and poloxamines.

Suitable binders impart cohesiveness to edible formulations. Materials suitable for use as binders in the solid dosage forms described herein include celluloses, microcrystalline dextrose, amylose, magnesium aluminum silicate, polysaccharide acids, bentonites, gelatin, polyvinylpyrrolidone/vinyl acetate copolymer, cross-povidone, povidone, starch, pregelatinized starch, tragacanth, dextrin, a sugar (e.g., sucrose, glucose, dextrose, molasses, mannitol, sorbitol, xylitol, lactose), a natural or synthetic gum (e.g., acacia, tragacanth, ghatti gum, mucilage of isapol husks), starch, PVP, larch arabinogalactan, Veegum®, PEG, waxes, and sodium alginate. Formulators skilled in the art can determine binder level for edible formulations.

Suitable lubricants or glidants for use in edible formulations include stearic acid, calcium hydroxide, talc, corn starch, sodium stearyl fumarate, alkali-metal and alkaline earth metal salts, stearic acid, sodium stearates, magnesium stearate, zinc stearate, waxes, Stearowet®, boric acid, sodium benzoate, sodium acetate, sodium chloride, leucine, PEG, methoxy-polyethylene glycol, propylene glycol, sodium oleate, glyceryl behenate, glyceryl palmitostearate, glyceryl benzoate, and magnesium or sodium lauryl sulfate.

Suitable diluents for use in edible formulations include sugars (including lactose, sucrose, and dextrose), polysaccharides (including dextrates and maltodextrin), polyols (including mannitol, xylitol, and sorbitol), and cyclodextrins. Non-water-soluble diluents are compounds typically used in the formulation of pharmaceuticals, such as calcium phosphate, calcium sulfate, starches, modified starches and microcrystalline cellulose, and micro cellulose (e.g., having a density of about 0.45 g/cm³, e.g., Avicel, powdered cellulose), and talc.

Suitable wetting agents for use in edible formulations include oleic acid, triethanolamine oleate, glyceryl monostearate, sorbitan monooleate, sorbitan monolaurate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, and vitamin E TPGS. Wetting agents include surfactants.

Suitable surfactants for use in the edible formulations described herein include docusate and its pharmaceutically acceptable salts, sodium lauryl sulfate, sorbitan monooleate, poly-oxyethylene sorbitan monooleate, polysorbates, poloxamers, bile salts, glyceryl monostearate, copolymers of ethylene oxide and propylene oxide, e.g., Pluronic® (BASF), and the like.

Suitable suspending agents for use in edible formulations include polyvinylpyrrolidone, PEG (having a molecular weight of about 300 to about 6000, or about 3350 to about 4000, or about 7000 to about 18000), vinylpyrrolidone/vinyl acetate copolymer (S630), sodium alginate, gums (e.g., gum tragacanth and gum acacia, guar gum, xanthans, including xanthan gum), sugars, celluloses, polysorbate-80, polyethoxylated sorbitan monolaurate, polyethoxylated sorbitan monolaurate, and povidone.

Suitable antioxidants for use in edible formulations include butylated hydroxytoluene (BHT), butyl hydroxyanisole (BHA), sodium ascorbate, Vitamin E TPGS, ascorbic acid, sorbic acid, and tocopherol.

The above-listed additives are merely exemplary types of additives that can be included in the disclosed edible formulations of the present invention. The amounts of such additives can be readily determined by one skilled in the art, according to the particular properties desired.

c. Beverage Formulations

In embodiments, disclosed A. muscaria extracts or compositions are provided in a beverage formulation for oral administration.

In embodiments, the disclosed A. muscaria compositions are used as a beverage, such as a drinkable liquid. In embodiments, the disclosed A. muscaria compositions are used in the manufacture of a beverage. In embodiments, the disclosed A. muscaria compositions are administered to a subject as a beverage. In embodiments, the disclosed A. muscaria compositions are provided as a powder, e.g., a beverage powder for mixing with a liquid. In embodiments, the disclosed A. muscaria compositions are provided as a ready to drink beverage. In embodiments, a ready to drink beverage is provided as a packaged beverage, such as in a prepared form, ready for consumption. In embodiments, the beverage provides any one or more of the benefits described herein to a subject who ingests said beverage. In embodiments, the beverage comprising a disclosed A. muscaria composition is prepared from a beverage powder. In embodiments, the beverage powder comprises a disclosed A. muscaria composition, e.g., an extract, compound, and analog thereof. The beverage powder can be added to a variety of liquids to introduce the nutritional benefits of a disclosed A. muscaria composition to the beverage. In embodiments, the beverage powder comprising a disclosed A. muscaria composition is added to a beverage comprising dairy. In embodiments, the beverage powder comprising a disclosed A. muscaria composition is added to another powder comprising dairy. Examples of dairy beverages, whether in liquid or powder form, include milk, chocolate milk, or hot chocolate. In embodiments, the beverage powder comprising a disclosed A. muscaria composition is added to a beverage comprising protein. In embodiments, the beverage powder comprising a disclosed A. muscaria composition is added to another powder comprising protein, such as a protein powder.

In embodiments, the beverage comprising a disclosed A. muscaria composition is a ready-to-drink beverage. In some embodiments the disclosed A. muscaria compositions will be incorporated into a ready-to-drink beverage. A ready-to-drink beverage is a beverage sold in a prepared form ready for consumption. Exemplary benefits provided by a ready to drink beverage comprising an A. muscaria composition include improvements in endurance and strength, promotion of calmness, relief from muscle soreness, and achievement of spiritual enlightenment. Improvements in such areas are described herein, such as above. In some embodiments the ready-to-drink beverage comprising the disclosed A. muscaria compositions is a dairy beverage. Examples of ready-to-drink dairy beverages include malted milk, chocolate milk, or strawberry milk. In some embodiments the ready-to-drink beverage comprising disclosed A. muscaria compositions is an herbal beverage. Examples of ready-to-drink herbal beverages include black tea, chamomile tea, or green tea. In some embodiments the ready-to-drink beverage comprising disclosed A. muscaria compositions is a carbonated beverage. Examples of ready-to-drink carbonated beverages include cola, carbonated water, or ginger ale. In some embodiments the ready-to-drink beverage is a fruit-based beverage. Examples of ready-to-drink fruit-based beverages include lemonade, smoothies, or fruit juice. In some embodiments the ready-to-drink beverage comprising disclosed A. muscaria compositions is an alcoholic beverage. Examples of ready-to-drink alcoholic beverages include beer, hard cider, or a cocktail.

In embodiments, the beverage formulation comprises L-theanine. L-theanine, an amino acid primarily found in tea leaves, particularly in green tea, has garnered attention for its potential health benefits, including promoting relaxation and reducing stress and anxiety levels by increasing alpha brain wave activity, which is associated with a state of wakeful relaxation (Kimura et al. Biological psychology 2007; 74 (1): 39-45). Additionally, some studies suggest that L-theanine may improve sleep quality by reducing stress and promoting relaxation before bedtime (id.). Hence, in some embodiments, beverage formulations comprising a disclosed extract in combination with L-theanine offer synergistic benefits that promote relaxation and improve sleep quality.

In embodiments, the beverage formulation comprises magnesium. Magnesium is an essential mineral involved in hundreds of biochemical reactions in the body, playing a crucial role in various physiological processes. Without being bound by theory, magnesium may have calming effects on the brain and nervous system, by regulating neurotransmitters (e.g., GABA) and reducing the activity of the stress hormone cortisol (Boyle et al. Nutrients 2017; 9 (5): 429). Additionally, magnesium supplementation has been shown to improve sleep onset, duration, and overall sleep quality (Arab et al. Biological Trace Element Research 2023; 201:121-128). Hence, magnesium may therefore promote feelings of relaxation and calmness and improve sleep quality when administered in combination with a disclosed extract.

In embodiments, the beverage formulation comprises an excipient. Non-limiting examples of excipients include tastants, flavoring agents, buffering agents, preservatives, stabilizers, binders, lubricants, dispersion enhancers, disintegration agents, sweeteners, and coloring agents.

In embodiments, the beverage formulation comprises a buffering agent. Non-limiting examples of suitable buffering agents include sodium citrate, magnesium carbonate, magnesium bicarbonate, calcium carbonate, and calcium bicarbonate.

In embodiments, the beverage formulation comprises a preservative. Non-limiting examples of suitable preservatives include antioxidants, such as alpha-tocopherol and ascorbate, and antimicrobials, such as parabens, chlorobutanol, and phenol.

In embodiments, the beverage formulation comprises a binder. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In embodiments, the beverage formulation comprises a lubricant. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In embodiments, the beverage formulation comprises a dispersion enhancer. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In embodiments, the beverage formulation comprises a disintegration agent. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. In some embodiments the disintegrant is an effervescent disintegrant.

In embodiments, the beverage formulation comprises a flavoring agent or a mixture of flavoring agents including natural or synthetic flavorants, such as flavoring oils, flavoring aldehydes, esters, alcohols, similar materials, and combinations thereof. Flavorants may include vanillin, spearmint oil, cinnamon oil, oil of wintergreen (methylsalicylate), peppermint oil, clove oil, anise oil, eucalyptus oil, citrus oils, fruit oils, essences, limonene, menthone, carvone, menthol, anethole, eucalyptol, anethole, eugenol, cassia, oxanone, a-irisone, propenyl guaiethol, thymol, linalool, benzaldehyde, cineole, cinnamaldehyde, cinnamaldehyde glycerol acetal (CGA), methone glycerol acetal (MGA), 3-I-menthoxypropane-1,2-diol, N-ethyl-p-menthan-3-carboxamine, N,2,3-trimethyl-2-isopropylbutanamide, and combinations thereof. Flavoring agent(s) may be present, individually or in total (if more than one flavoring agent is included), in disclosed formulations in an amount ranging from about 0.1 wt % to about 10 wt % (calculated as the total weight of flavoring agent(s) in the formulation divided by the total weight of the formulation).

In embodiments, the beverage formulation comprises a flavoring agent, which may be a flavor. A flavor may be any of a liquid flavor, a powder flavor, a powdered encapsulated flavor, a spray-dried flavor, an emulsion flavor, a plant, fruit, vegetable, or other extraction-based flavor, a seasoning, an organic flavor, a natural flavor, a non-GMP flavor, a clean label flavor, a concentrated flavor, a savory flavor, a sweet flavor, a masking flavor, and the like, including combinations thereof. Flavors may be produced using methods known to those in the art, or obtained from flavor suppliers as known in the art, for example from Gold Coast Ingredients, Inc. (GCI) (Commerce, California). Flavoring agents include, for example, synthetic flavor oils and flavoring aromatics; natural oils; extracts from plants, leaves, flowers, and fruits; and combinations thereof. In embodiments, the beverage formulation comprises a flavor of plum, prune, date, currant, fig, grape, raisin, cranberry, pineapple, peach, nectarine, banana, apple, pear, guava, apricot, Saskatoon berry, blueberry, plains berry, prairie berry, mulberry, elderberry, Barbados cherry (acerola cherry), choke cherry, chocolate, vanilla, caramel, coconut, olive, raspberry, strawberry, huckleberry, loganberry, dewberry, boysenberry, kiwi, cherry, blackberry, honey dew, green tea, cucumber, quince, buckthorn, passion fruit, sloe, rowan, gooseberry, pomegranate, persimmon, mango, rhubarb, papaya, litchi, lemon, orange, lime, tangerine, mandarin and grapefruit juices, or any combination thereof.

In embodiments, the beverage formulation comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and hydrogenated starch hydrolysates.

In embodiments, the beverage formulation comprises a coloring agent. Non-limiting examples of suitable color agents include food, drug and cosmetic colors (FD&C), drug and cosmetic colors (D&C), and external drug and cosmetic colors (Ext. D&C).

d. Topical Formulations

In embodiments, the disclosed A. muscaria extracts or compositions are provided for topical administration. In embodiments, topical or transdermal administration of a disclosed A. muscaria composition provides skin pain relief, pruritus, rejuvenation and moisturization, and immune enhancement.

In embodiments, a disclosed A. muscaria compositions further includes a topical delivery system for topical transdermal delivery. An exemplary topical delivery system is a transdermal delivery device (“patch”) containing the active agents. Such transdermal patches may be used to provide continuous or discontinuous infusion of the compounds of embodiments of the present invention in controlled amounts. Such patches may be constructed for continuous, gradual, pulsatile, or on demand delivery of pharmaceutical agents, for example. A “patch” within the meaning of the invention may be simply a medicated adhesive patch, i.e., a patch impregnated with a pharmaceutical composition in accordance with an embodiment of the invention for application onto the skin. Thus, a patch may be a single-layer or multi-layer drug-in-adhesive patch, wherein the one or more adhesive layers also contain the active agents.

A patch may also be a “matrix” (or “monolithic”) patch, wherein the adhesive layer surrounds and overlays the drug layer (wherein a solution or suspension of the active agents is in a semisolid matrix). A “reservoir” patch may also be used, comprising a drug layer, typically as a solution or suspension of the active agents in a liquid compartment (i.e., the reservoir), separate from an adhesive layer. For example, the reservoir may be totally encapsulated in a shallow compartment molded from a drug-impermeable metallic plastic laminate, with a rate-controlling membrane made of vinyl acetate or a like polymer on one surface. A patch also may be part of a delivery system, for instance used with an electronic device communicatively coupled to the mobile device of a user, and coupled with a mobile application (e.g., to control the delivery rate from the reservoir, and optionally to provide information about delivery back to the app or user). Various transdermal patch technologies may be accordingly utilized, as known in the art.

In one example, a transdermal patch that may be used includes a self-contained module having a built-in battery that produces a low-level electric current to heat the skin and deliver a prescribed dose of a composition of an embodiment of the invention, wherein a therapeutically effective amount of the composition crosses the skin and enters the underlying tissue, so as to produce a therapeutic effect. Such a transdermal delivery device may, for example, comprise an adhesive layer, a protective film, a drug-containing reservoir (for the pharmaceutical compositions of embodiments of the invention), a heating coil, a battery, a hardware board, optionally all within a device holder, and optionally, functionally coupled to a device which is able to control drug delivery (e.g., a mobile device such as a smartphone) using a downloadable application. Such devices may, for instance, additionally shut off drug delivery automatically when a prescribed dose has been administered or may shut off automatically upon reaching a certain temperature or defined time. Such transdermal devices may be reusable or disposable.

Other release system technologies that can be used with the pharmaceutical compositions of embodiments of the invention include aromatic patches, hydrogel patches, iontophoretic patches, polyurethane patches, Geltronik patches, hyaluronic acid patches, microneedle patches, and wet wipes. Transdermal formulations in accordance with this embodiment of the invention may be applied to skin daily, such as twice to four times a day, for example, by applying a respective patch directly to the skin. Formulations for delivery through transdermal delivery devices are similar to the creams and ointments described above, except for the oily compounds for forming the creams and ointments, and the thickener for forming a gel, are not needed. Water may therefore be a sufficient excipient and the formulations can have a higher water content than the creams and appointments described above. Emulsifiers would only be required if oil based ingredients are included, such as oil-based anti-inflammatoireanti-oxidants, and/or humectants, for example. As above, the formulations may include from about 1 to about 10 micrograms per milliliter of A. muscaria extract, prepared in accordance with the Tancowny process or other processes described above or known in the art, as described above. Other amounts may be provided. Any one or more of the anti-inflammatoires, antioxidants, humectants, and preservatives with respect to the creams and ointments above may be included in the transdermal formulations. In addition, a stabilizer, a solubilizer, and/or a permeation enhancing agent may be provided, as is known in the art.

Pharmaceutical compositions may be prepared as a topical dosage form. Topical dosage forms include transmucosal and transdermal formulations, such as aerosols, emulsions, sprays, ointments, salves, gels, pastes, lotions, liniments, oils, patches, and creams. For such formulations, penetrants and carriers can be included in the pharmaceutical composition. Penetrants are known in the art, and include, for transmucosal administration, detergents, bile salts, and fusidic acid derivatives. For transdermal administration, carriers which may be used include Vaseline®, lanolin, PEG, alcohols, transdermal enhancers, and combinations thereof.

In embodiments, the disclosed compositions can be combined with other active agents, to form novel combined therapeutic formulations. Embodiments for skin care, dermatology, oral care, and cosmetic applications therefore would include skin patches, skin creams, hair loss patches, cold sore patches, mouth ulcer patches, scar reducers, hyper-hydrosis patches, skin protection patches, sun patches, protective patches, eye relax masks, pediatric teething gels, diaper sprays, menstrual pain or cramp warming patches, tension patches, anti-aging patches and masks, eye contour and eye bag patches, skin hydration patches, fat and cellulite patches, firming patches, body wraps, venopatches, nasal congestion patches, insect repellant patches, insect bite patches, foot plasters/cushions, hot/cold contrast therapy hydrogel and cold hydrogel patches, heating patches and skin wraps, and oral fluids, gels, and sprays.

In other embodiments, the disclosed compositions can be combined with other compounds such as vitamins, antioxidants, amino acids, probiotics, natural herbs or plant extracts, and other food or dietary supplements, to form patches, masks, wraps, creams, gels, oral films, or the like, for various purposes such as for energy, reduced fatigue, or improved mental and physical performance (e.g., with guarana, amino acids, and vitamins), for improved performance and reduced gastrointestinal discomfort during exercise (e.g., with L-leucine, L-isoleucine, L-valine, Vitamin B1, and Vitamin B5), for osteoporosis or function of bones and teeth (e.g., with vitamins C, D3, and K2), for sexual dysfunction, improved sexual vigor, or fertility (e.g., with vitamins, amino acids, and plant extracts such as maca root, Ginkgo biloba, tributes terrestris, minus pinaster, muir puma, damiana, or catuaba), for improved circulation or to sooth heavy legs (e.g., with vitas vinifera extract and bioflavonoids), for relaxation and sleep (e.g., with melatonin, passionflower, Californian poppy, and other herbal ingredients), or to relieve muscle and joint pain or trauma, (e.g., with hemp seed oil, arnica, and harpagophytum).

e. Vaporizer Formulations

In embodiments, compounds and extracts are formulated for administration by vaporization. Suitable vaporizer formulations may comprise the compounds and/or extracts described herein, a base liquid comprising any of propylene glycol (PG), vegetable glycerin (VG), polyethylene glycol (PEG), and optionally water and ethanol (where the ethanol may be an alcoholic drink or spirit, including vodka); and optionally a flavorant. In embodiments, the optional flavorants include flavor concentrates known to those of skill, for example, flavor concentrates that replicate different food and drink flavors.

Common base liquid proportions include 50:50 (PG/VG), 30:70 (PG/VG), and 20:80 (PG/VG). However, said proportions should not be construed as limiting, as described above, the base liquid may contain anywhere from 1% to 100% PG, and/or anywhere from 1% to 100% VG. In embodiments, one may also substitute PG or VG for polyethylene glycol (PEG), such that the base liquid comprises anywhere from 1% to 100% PEG, with the remaining base liquid, if applicable, comprising a proportion of PG and/or VG. In embodiments, the base liquid, compounds and/or extracts, and optional flavorant may be combined via means known to those of skill in a container suitable for the resultant formulation. Once combined, the base liquid, compounds and/or extracts, and optional flavorant may be agitated to form a uniform mixture by an operator, or via paddles, arms, or other agitation means within the container.

In embodiments, the resultant vaporizer formulation comprises compounds and/or extracts in a proportion of between 1% v/v to 50% v/v, base liquid in a proportion of between 40% v/v to 90% v/v, and optionally one or more flavorants in a proportion of between 1% v/v to 50% v/v, where each range is inclusive.

In embodiments, the formulations may be utilized with any vaporizer device known to those of skill, including a device that is mouth-to-lung or direct-to-lung, a device that uses single-use, disposable pods; a device that uses refillable pods, a modified or “mod” pod device, including a closed pod system and an open pod system; a pen device that can be refilled with the formulations disclosed herein, including simple refillable pens, such as fixed voltage pens, vape cartridges or carts, e.g., standard 0.5 mL or 1.0 ml cartridges, variable voltage pens, and variable temperature pens; and modified pens (mods) that are custom-crafted by the user, including regulated mods (containing a circuit board) and unregulated mods (not containing a circuit board), tube mods, box mods, and mechanical mods (mechs); a disposable, single-use pen device; an e-cigarette device, a rechargeable e-cigarette device, a cigalike device, including a disposable cigalike and a refillable cigalike; an e-cigar, an e-pipe, and a heat-not-burn device.

### E. EXAMPLES

### Example 1: Extraction of Muscimol from Amanita Muscaria Mushrooms

The FIGURE depicts an example of an improved process for extracting muscimol from Amanita muscaria according to embodiments described herein, wherein such improved process is a process to produce A. muscaria extract at scale, such as at kilogram scale.

In the initial extraction step (100), 5 kg Amanita muscaria mushrooms were mixed with 70 L water in a 110 L jacketed tank (104) fitted with an overhead mixer. A heater (104) was used to heat the water/mushroom mixture to 80° C. for 10 minutes. A diaphragm pump (106) was used to transfer the water/mushroom mixture to the filtration apparatus (200), which consists of a dual filter housing containing two 200-micron sock filters (202). The water/mushroom mixture was then filtered to remove solid material, resulting in a liquid extract comprising muscimol and ibotenic acid. A Foxtrot 300 filter (Delta-9 Technologies, Hilton Head, SC) was used for the filtration step. Aqueous hydrochloric acid (HCl) was added in an amount sufficient to reduce the pH of the extract to 2.6. Then, in the decarboxylation step (300) the extract was refluxed to facilitate the decarboxylation of ibotenic acid to muscimol. Specifically, the extract was transferred in batches into two round-bottom flasks (302 and 304), each fitted with a reflux condenser connected to a chiller (306). Heating mantles were used to bring each flask to reflux temperature (ca. 96° C.) for four hours. After the reflux, the extract was prepared for formulation and packaging (400). First, an amount of 1 M aqueous sodium hydroxide (NaOH) sufficient to raise the pH to about 4.0 was added to the refluxed extract. The total volume of extract was transferred to a 110 L jacketed tank fitted (402) with an overhead mixer. At this stage, flavoring agents and preservatives may be added to the extract. In one example, corn flavoring agent, sodium benzoate, and potassium sorbate were added to the extract. A peristaltic filling pump (404) was used to fill the extract into amber-colored 30-mL glass vials (406), which were then capped with dropper caps (408). The bottles were shrink-wrapped (410), labeled (412), and cartoned (414). The total extraction efficiency was typically 85-90%.

### Example 2: Chemical Fingerprinting of Ground Mushroom Powder

Purpose: This example provides an exemplary procedure for evaluating the concentrations of stizolobic acid and stizolobinic acid in ground mushroom samples by HPLC-MS/MS normal phase chromatography prior to their use in a disclosed extraction method.

Instrumentation: Agilent 1290 Infinity II with 6470 triple quadrupole mass spectrometer, fitted with an Agilent Poroshell 120 HILIC-Z, 2.1×10⁰ mm, 2.7 μm (P.N. 685775-924, Lot No. B19528), or a UHPLC Guard 3 pk. InfinityLab Poroshell 120 HILIC-Z 2.1×5 mm, 2.7 μm (P.N. 821725-947).

Certified Reference Materials: Stizolobic acid (SA1) (CanAM Bioresearch Inc.) (CAS No: 15911-87-2) and Stizolobinic acid (SA2) (CanAM Bioresearch Inc.) (CAS No: 15911-87-2)

Apparatus and materials required: 1. Vortex mixer; 2. Sonicator bath; 3. Centrifuge; 4. 50 mL centrifuge tubes; 5. 1.8 mL HPLC autosampler vials; 6. Formic Acid (FA), LC-MS grade (Fisher Scientific Lot No. 219026); 7. Acetonitrile (ACN), LC-MS grade (Fisher Scientific Lot No. 218711); 8. Ammonium Formate (>99%), LC-MS LiChropur grade (Sigma-Aldrich 7022-25G-F); 9. Type I water in house Sartorius Water purified system

### Solution Preparation:

Reference standard stizolobic acid (SA1) stock solution: 1.18 mg of stizolobic acid (CAS No: 15911-87-2) was weighed into a 10 mL volumetric flask and made up to 10 ml of water and sonicated for 3 minutes to yield a clear solution. This was the primary stock solution for calibration curve solutions which is equivalent to 118 ppm.

Reference standard stizolobinic acid (SA2) stock solution: 1.13 mg of stizolobinic acid (CAS No: 15911-87-2) was weighed into a 10 mL volumetric flask and made up to 10 mL of water and sonicated for 3 minutes to yield a clear solution. This was the primary stock solution for calibration curve solutions which is equivalent to 113 ppm.

Calibration: A mixture of calibration 20 ppm SA1/SA2 from the 118 ppm SA1 and the 113 ppm SA1 was prepared in 50% ACN: 0.01% FA. From the solution the calibration standard of concentrations 10 ppm, 5 ppm, 2 ppm, 1 ppm, 0.5 ppm, 0.2 ppm, 0.1 ppm, 0.05 ppm and 0.02 ppm were prepared by serial dilution.

Sample Preparation: 0.15 gram of ground mushroom sample was weighed with a 4 decimal place balance after being equilibrated from freezer to room temperature in a 50 mL centrifuge tube. 15 mL type I water was added. The mixture was vortexed for 1 minute, sonicated for 15 minutes, and vortexed for another minute. After extraction, the solution was centrifuged at 3500 rpm for 10 min. 1.5 mL of supernatant aliquot of this mixture was filtered with a 0.2 μm filter and transferred to an autosampler vial. This solution was labeled as Mushroom-Sample-1 and so on for other samples.

### HPLC Parameters:

Mobile Phase A: 10 mM ammonium formate: 0.01% formic acid
Mobile Phase B: 10 mM ammonium formic acid: 90% Acetonitrile: 0.01% formic acid
Column: Agilent Poroshell 120 HILIC-Z, 2.1×10⁰ mm, 2.7 μm
Detector: Agilent 6470 Triple Quadruple Mass Spectrometer
Injection volume: 1 μL
Temperature: 30° C.
Flow: 0.25 mL/min

Results: TABLE 7 shows the concentrations of stizolobic acid and stizolobinic acid in an exemplary sample of ground mushroom, obtained according to the procedure described above. These results show that the mushroom extract contained less than less than 50 μg/g of both stizolobic acid and stizolobinic acid, which Applicant has determined is indicative of the mushroom sample comprising sufficiently pure Amanita muscaria for use in a disclosed extraction method (e.g., which does not comprise substantial amounts of Amanita pantherina).

TABLE 7
Measured concentrations of stizolobic acid and
stizolobinic acid in an exemplary ground
mushroom sample.

| Stizolobic Acid (SA1) | | | Stizolobinic Acid (SA2) | | |
|---|---|---|---|---|---|
| LOD* | LOQ** | Result | LOD* | LOQ** | Result |
| (μg/g) | (μg/g) | (μg/g) | (μg/g) | (μg/g) | (μg/g) |
| 0.5 | 1.0 | 31.6 | 0.2 | 0.5 | 5.4 |

LOD* = Level of Detection;
LOQ** = Level of Quantification

### Example 3: Beverage Formulations Comprising A. Muscaria Extracts

Amanita muscaria extracts produced according to disclosed methods were formulated into beverages, which were subjected to HPLC-MS/MS analysis to determine the concentrations of muscimol, muscarine, and ibotenic acid.

Beverage Formulation 1:8 oz beverage comprising Amanita muscaria extract, mango flavoring, L-theanine, and magnesium.

TABLE 8
Concentrations of Alkaloids in Beverage Formulation 1,
Determined by HPLC-MS/MS

| Compound | Result (μg/g) |
|---|---|
| Ibotenic Acid | ND* |
| Muscarine | ND* |
| Muscimol | 1.2 |

*ND = not detected

Beverage Formulation 2:8 oz beverage comprising Amanita muscaria extract, mango flavoring, and magnesium.

TABLE 9
Concentrations of Alkaloids in Beverage Formulation 1,
Determined by HPLC-MS/MS

| Compound | Result (μg/g) |
|---|---|
| Ibotenic Acid | ND* |
| Muscarine | ND* |
| Muscimol | 1.14 |

*ND = not detected

Beverage Formulation 3:8 oz beverage comprising Amanita muscaria extract, mango flavoring, and L-theanine.

TABLE 10
Concentrations of Alkaloids in Beverage Formulation 1,
Determined by HPLC-MS/MS

| Compound | Result (μg/g) |
|---|---|
| Ibotenic Acid | ND* |
| Muscarine | ND* |
| Muscimol | 1.24 |

*ND = not detected

### Example 4: Ingredient List for Exemplary Skin Cream Formulation Containing an A. muscaria Extract

A skin cream formulation for topical administration comprising an A. muscaria extract was prepared with the following ingredients.

International Nomenclature of Cosmetic Ingredients
(INCI) Name (Trade Name)

Water
Aloe Barbadensis Leaf Juice
Carbomer
Butylene glycol
Sodium hyaluronate
Squalane
Shea butter White Refined
Octyldodecanol
Ethylhexyl Hydroxystearate
Stearic Acid
Polyglyceryl-2 Stearate (and) Glyceryl Stearate (and)
Stearyl Alcohol
Cetyl alcohol
Dimethicone
Potassium Cetyl Phosphate
Hydroxyethyl Acrylate/Sodium Acryloyldimethyl
Taurate Copolymer
Colloidal Oatmeal (and) Leuconostoc/Radish Root
Ferment Filtrate
Amanita Muscaria Extract - present in formulation at 2.5%
Water (and) Panax Ginseng Root Extract (and)
Leuconostoc/Radish Root Ferment Filtrate
Ceramide NP (ceramide 3), Ceramide AP (ceramide 6),
Ceramide EOP (ceramide 9), Phytosphingosine,
Cholesterol, Sodium lauroyl lactylate, Carbomer,
Xanthan gum, Water, Phenoxyethanol, Ethylhexylglycerin
Dimethicone
Phenoxyethanol (and) Ethylhexylglycerin
Fragrance (AM54-00376 Lotus & Pomegranate )

### Example 5: Formulation of an Aqueous or Oil Based Liquid Suspension or Solution

Liquid suspensions or solutions, with amounts per 1.0 ml are as follows:

| Ingredient | Amount | |
|---|---|---|
| A. muscaria extract | 20 | mg |
| Preservative | 3.0 | mg |
| Excipient | to 1.0 | mL |

A. muscaria extract of the disclosure is mixed with an excipient, such as distilled water, an alcohol such as ethanol, or a food grade carrier oil, such as MCT oil, coconut oil, or hemp seed oil, and optionally a polyol (e.g., vegetable glycerin) and/or a lecithin (e.g., soy or sunflower lecithin). A preservative optionally will be included (e.g., sodium bisulfate, sodium citrate, and/or citric acid). Additional agents may be included if desired, e.g., flavors, sweeteners (including artificial sweaters), vitamins, active ingredients (e.g., antioxidants or anti-inflammatoires), herbal extracts, essential oils, and/or any one or more of such other agents as described herein. Sufficient further excipient is then added to produce the required volume. Suspensions may be prepared in volumes of 5 mL, 10 mL, 25 mL, 30 mL, 50 mL, 100 mL, or such other total volumes as practical for research use or for sale as a pharmaceutical or OTC preparation, or a nutraceutical preparation or dietary supplement. Depending on unit dosage volume and total volume, suspensions may be used to prepare dropper bottles (e.g., 30 mL/1 oz. bottles) or fine mist spray (i.e., oral spray) bottles (e.g., 10 ml bottles). Liquid suspensions of this Example also can be used to prepare softgel capsules, ampoules, or other single unit dosage forms, through methods herein disclosed or known to those of skill.

### Example 6: Formulation of an Aqueous Liquid Solution

Water-based liquid suspensions, with amounts per 100 mL are as follows:

| Ingredient | Amount | |
|---|---|---|
| A. muscaria extract | 500 | mg |
| Melatonin | 2.5 | mg |
| L-theanine | 100 | mg |
| 5-hydroxytryptophan | 50 | mg |
| Preservative | 3.0 | mg |
| Excipient | to 100 | mL |

A. muscaria extract is mixed with water as the excipient (e.g., distilled water, deionized water, reverse osmosis or other purified water, and the like), together with one or more additional active agents for sleep, such as melatonin, L-theanine, and/or 5-hydroxytryptophan (5-HTP). A preservative optionally will be included (e.g., sodium bisulfate, sodium citrate, and/or citric acid). Additional agents may be included if desired, e.g., flavors, sweeteners, vitamins, and/or any one or more of such other agents as described herein. Sufficient further excipient is then added to produce the required volume. Suspensions may be prepared in volumes of 5 mL, 10 mL, 25 mL, 30 mL, 50 mL, 100 mL, or such other total volumes as practical for research use or for sale as a pharmaceutical or OTC preparation, or a nutraceutical preparation or dietary supplement. Depending on unit dosage volume and total volume, suspensions may be used to prepare dropper bottles (e.g., 30 mL/1 oz. bottles) or fine mist spray (i.e., oral spray) bottles (e.g., 10 mL bottles). Liquid suspensions of this Example also can be used to prepare softgel capsules, ampoules, or other single unit dosage forms, through methods herein disclosed or known to those of skill.

### Example 7: Formulation of a Liquid Suspension or Solution, Such as a Tincture

Liquid suspensions or solutions, with amounts per 1.0 mL are as follows:

| Ingredient | Amount | |
|---|---|---|
| A. muscaria extract | 80 | mg |
| Alpha lipoic acid (antioxidant) | 40 | mg |
| Sodium carboxymethyl cellulose (11%) | 12.5 | mg |
| Microcrystalline cellulose (89%) | 10 | mg |
| Sweetener | 0.35 | g |
| Sodium benzoate | 2.5 | mg |
| Flavor and Color | | q.v. |
| Excipient | to 1.0 | mL |

A. muscaria extract is measured out (blended and passed through a mesh sieve if dry), and then mixed with a previously made solution of microcrystalline cellulose and sodium carboxymethyl cellulose in excipient (e.g., ethanol, for a tincture, or purified water). The sodium benzoate, flavor, and color are diluted with excipient and added with stirring. Sweetener (e.g., sucrose or sucralose) may be added if desired. Additional agents may be included, e.g., GABA or another GABA agent, vitamins, other active ingredients such as antioxidants or anti-inflammatoires, herbal extracts, and essential oils. Sufficient further excipient is then added to produce the required volume. Suspensions and solutions may be prepared in volumes of 5 mL, 10 mL, 25 mL, 30 mL, 50 mL, 100 mL, or such other total volumes as practical for research use or for sale as a pharmaceutical or OTC preparation, a nutraceutical preparation or dietary supplement, or a natural product. Depending on unit dosage volume and total volume, liquid formulations may be used to prepare dropper bottles (e.g., 30 mL/1 oz. bottles) or fine mist spray (i.e., oral spray) bottles (e.g., 10 ml bottles). Liquid formulations of this Example also can be used to prepare filled softgel capsules, ampoules, or other single unit dosage forms, using methods herein disclosed or known to those of skill.

### Example 8: Formulation of Tablets

A tablet is prepared using the ingredients below:

| Ingredient | Quantity (mg/tablet) |
|---|---|
| A. muscaria extract | 120 |
| Cellulose, microcrystalline | 170.0 |
| Colloidal silicon dioxide | 10.0 |
| Stearic acid | 7.5 |

The ingredients are blended and compressed to form tablets. It will be appreciated in all exemplary formulations below involving a solid form, (e.g., a tablet or capsule) even when not explicitly stated, that the extract will be obtained as a dry powder.

### Example 9: Alternate Formulation of Tablets

Scorable tablets are prepared as follows:

| Ingredient | Quantity (mg/tablet) |
|---|---|
| A. muscaria extract | 120 |
| Starch | 45.0 |
| Microcrystalline cellulose | 35.0 |
| PVP (as 10% solution in water) | 4.0 |
| Sodium carboxymethyl starch | 4.5 |
| Magnesium stearate | 0.5 |
| Talc | 1.0 |

A. muscaria extract, starch and cellulose are passed through a No. 20 mesh U.S. sieve and mixed thoroughly. The solution of polyvinylpyrrolidone (PVP) is mixed with the resultant powders, which are then passed through a 16 mesh U.S. sieve. The granules so produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets. Tablets are scored to provide the ability to create equal half doses.

### Example 10: Formulation of Capsules

Capsules are made as follows:

| Ingredient | Quantity (mg/capsule) |
|---|---|
| A. muscaria extract | 50 |
| Starch | 119.0 |
| Magnesium stearate | 1.0 |

A. muscaria extract, cellulose, starch, and magnesium stearate are blended, passed through a No. 20 mesh U.S. sieve, and filled into hard or soft gelatin capsules.

### Example 11: Formulation of Capsules with Additional Active Agent(s)

Capsules are made as follows:

| Ingredient | Quantity (mg/capsule) |
|---|---|
| A. muscaria extract | 100 |
| Cannabinoid(s) | 50.0 |
| Terpene(s) | 10.0 |
| Starch | 119.0 |
| Magnesium stearate | 1.0 |

A. muscaria extract, cellulose, starch, and magnesium stearate are blended, passed through a No. 20 mesh U.S. sieve, and filled into hard or soft gelatin capsules. The cannabinoid(s) and/or terpene(s) (one or the other or both may be added, including combinations of multiple such compounds) will be as generally understood in the art.

Additional active agents may include one or more cannabinoids and/or terpenes.

“Cannabinoid” refers to any one of the class of compounds that act on cannabinoid receptors or the endocannabinoid system. In embodiments, cannabinoids include tetrahydrocannabinol (THC, including delta-9 and delta-8 THC), cannabidiol (CBD), cannabichromene (CBC), cannabidinodiol (also known as cannabinodiol) (CBND, CBDL), cannabielsoin (CBE), cannabicyclol (CBL), cannabicitran (CBT), cannabitriol (CBT), cannabivarin (CBV), cannabigerol monomethyl ether (CBGM), cannabidiphorol (CBDP), tetrahydrocannabiphorol (THCP), and iso-tetrahydrocannabinol (iso-THC), as well as their acidic forms, their propyl, methyl, and ethyl homologues, and the acid forms of those homologues forms (i.e., their acidic propyl, acidic methyl, and acidic ethyl homologue forms). Cannabinoids further include cannabinoid glycoside, acetylated, and acetylated cannabinoid forms, e.g., as described in U.S. patent application Ser. Nos. 16/110,728 and 16/110,954.

“Terpene” refers to any of the class of organic hydrocarbon isoprene polymers (isoprenoids) constituted by one or more repeating units of the five-carbon building block known as the isoprene unit (i.e., 2-methyl-1,3-butadiene, having the molecular formula C₅H₈), and including such terpenes as those structurally found in linear chains or in rings, and those having any number of isoprene units, i.e., whether as hemiterpenes (one unit), monoterpenes (two), sesquiterpenes (three), diterpenes (four), sesterterpenes (five), triterpenes (six), sesquiterpenes (seven), tetraterpenes (eight), or polyterpenes (nine or more). Terpenes are contemplated as being either derived from a botanical source, whether from cannabis or another plant, or synthetic. In embodiments, the terpenes are selected from the group consisting of alpha-bisabolol, beta-caryophyllene, camphene, carene, caryophyllene oxide, alpha-humulene, fenchol, guaiene, guaiol, limonene, linalool, myrcene, nerolidol, ocimene, alpha-phellandrene, alpha-pinene, beta-pinene, alpha-terpinene, gamma-terpinene, terpineol, and terpinolene.

### Example 12: Formulation of Capsules with Additional Active Agent(s)

Capsules are made as follows:

| | Quantity |
|---|---|
| Ingredient | (mg/capsule) |
| A. muscaria extract | 7.5 |
| Antidepressant, anxiolytic, or | 50.0 |
| GABA agent | |
| Starch | 119.0 |
| Magnesium stearate | 1.0 |

A. muscaria extract, cellulose, starch, and magnesium stearate are blended, passed through a No. 20 mesh U.S. sieve, and filled into hard or soft gelatin capsules.

Additional active agents may include antidepressants, anxiolytics, and/or GABA agents.

An antidepressant or anxiolytic may be any pharmaceutical agent known to act as such by one of skill (e.g., SSRIs, serotonin-norepinephrine reuptake inhibitors (SNRIs), tricyclic antidepressants (TCAs), atypical antidepressants, benzodiazepines, buspirone, etc.).

“GABA agent” refers generally to a compound that modulates the activity of a GABA receptor relative to the activity of the GABA receptor in the absence of the compound, or that otherwise elicits an observable response upon contacting a GABA receptor, including one or more subtypes. GABA agents useful in the disclosed methods include agents that modulate GABA receptor activity (as an agonist, partial agonist, antagonist, or allosteric modulator). In embodiments, GABA receptor activity is reduced by at least about 50%, or at least about 75%, or at least about 90%. In further embodiments, GABA receptor activity is reduced by at least about 95%, or by at least about 99%. In other embodiments, GABA receptor activity is enhanced by at least about 50%, or at least about 75%, or at least about 90%. In additional embodiments, GABA receptor activity is increased by at least about 95% or at least about 99%.

Exemplary non-limiting GABA agents include (besides muscimol) baclofen, arbaclofen placarbil, bicuculline, lesogaberan, indillon, phenibut, primidone, pentetrazol, valproic acid, progabide, zaleplon, SGS-742, AZD 3353, clomethiazole, tramiprosate, gaboxadol (4,5,6,7-tetrahydroisoxazolo (5,4-c)pyridin-3-ol (THIP)), Thio-THIP, THIA, isoguvacine, adipiplon, cis-aminocrotonic acid (CACA), CGP 642103 (CAS 200402-50-2), and 1,2,5,6-tetrahydropyridine-4-yl methyl phosphinic acid (TPMPA). Additional non-limiting examples of GABA agents include those in U.S. Pat. Nos. 6,503,925; 6,218,547; 6,399,604; 6,646,124; 6,515,140; 6,451,809; and those in U.S. Patent Application Pub. Nos. 2019/0321341, 2005/0014939; 2004/0171633; 2005/0165048; 2005/0165023; 2004/0259818; and 2004/0192692; as well as such others as will be known to those of skill. In embodiments, a GABA agent will be GABA (γ-aminobutyric acid).

### Example 13: Formulation of Capsules with Additional Active Agent(s)

Capsules are made as follows:

| | Quantity |
|---|---|
| Ingredient | (mg/capsule) |
| A. muscaria extract | 6.0 |
| Analgesic or anti-inflammatory | 125.0 |
| Palmitoylethanolamide (PEA) | 50 |
| | mg |
| Starch | 119.0 |
| Magnesium stearate | 1.0 |

A. muscaria extract, cellulose, starch, and magnesium stearate are blended, passed through a No. 20 mesh U.S. sieve, and filled into hard or soft gelatin capsules.

Additional active agents may include one or more analgesics or anti-inflammatories.

An analgesic or anti-inflammatory would be any agent known to act as such (including both OTC and prescription medications) by one of skill (e.g., aspirin, acetaminophen, ibuprofen, naproxen, prescription NSAIDs, etc.).

PEA, an endogenous fatty acid amide and nuclear factor agonist shown to exert a variety of biological effects relating to chronic inflammation and pain, may be optionally included.

### Example 14: Formulation of Suspension

Suspensions are made as follows:

| Ingredient | Amount | |
|---|---|---|
| A. muscaria extract | 120 | mg |
| Xanthan gum | 4.0 | mg |
| Sodium carboxymethyl cellulose (11%) | 50.0 | mg |
| Microcrystalline cellulose (89%) | 50.0 | mg |
| Sucrose | 1.75 | g |
| Sodium benzoate | 10.0 | mg |
| Flavor and color (optional) | | q.v. |
| Purified water | To 5.0 | mL |

The A. muscaria extract, sucrose and xanthan gum are blended, passed through a No. 10 mesh U.S. sieve, and then mixed with a solution of the microcrystalline cellulose and sodium carboxymethyl cellulose in water. The sodium benzoate and optional flavor and color are diluted with water and added with stirring. Sufficient water is added to produce the required volume.

### Example 15: Formulation of Intravenous Solution

An intravenous formulation may be prepared as follows:

| Ingredient | Amount | |
|---|---|---|
| A. muscaria extract | 500 | mg |
| Isotonic saline | 1,000 | mL |

A. muscaria extract is dissolved in appropriate solvent as will be understood by those of skill; isotonic saline is used in this Example, but it will be appreciated that other solvents may be used, and additional active or inactive ingredients such as preservatives may be added, as otherwise described above, and within the general knowledge of the art.

### Example 16: Formulations of Injectable Form

Injectable formulation (e.g., for subcutaneous, intramuscular, intraperitoneal, or intravenous delivery) may be prepared as follows:

| Ingredient | Amount |
|---|---|
| A. muscaria extract | 1,000 mg |
| DMSO | 5 mL |
| TEG | 500 mL |
| Saline (1% cremophor) | To 1 L |

A. muscaria extract is dissolved in dimethyl sulphoxide (DMSO) in proportions of 1 g to 0.5 mL. Solution is brought to 37° C. and vortexed for 3-5 minutes. Tetraethyleneglycol (TEG) in the amount of 5 mL is added, and solution is returned to 37° C. and vortexed again for 3-5 mins. Solution is mixed 1:1 with saline containing 1% cremophor to prevent precipitation. Final solution will be at 10 mg/mL active ingredients in 49.5% TEG, 49.5% saline, 0.5% DMSO, and 0.5% cremophor. Injection may be by any suitable means, e.g., bolus injection, IV infusion, or subcutaneous infusion, for example using a drug delivery device comprising a reservoir and a pump mechanism, configured for subcutaneous administration, and which may optionally contain a user interface or be coupled to a device with a user interface such as a smartphone.

### Example 17: Formulation of Topical Form

A topical formulation for transdermal administration may be prepared as follows:

| Ingredient | Amount (mg) |
|---|---|
| A. muscaria extract | 120 |
| Emulsifying wax | 30.0 |
| Liquid paraffin | 20.0 |
| White soft paraffin | To 100 |

The white soft paraffin is heated until molten. The liquid paraffin and emulsifying wax are incorporated and stirred until dissolved. A. muscaria extract is added and stirring is continued until dispersed. The mixture is then cooled until solid.

It will be readily appreciated that topicals may contain any number of additional ingredients, according to the teachings herein and skill in the art. For example, in one exemplary embodiment, a topical comprises compound or an A. muscaria extract, aloe barbadensis leaf juice, parfum, Aleurites moluccanus seed oil, Simmondsia chinensis (jojoba) seed oil, polawax NF-PA-MH, rosa canina fruit oil, Theobroma cacao (cocoa) seed butter, nigella sativa seed extract, glycerin, hibiscus rosa-sinensis flower extract, Lavandula angustifolia (lavender) oil, citric acid, Raphanus sativus (radish) root extract.

### Example 18: Formulation of Transdermal Delivery Form

A formulation for a transdermal delivery device may be prepared as follows:

| Ingredient | Amount (mg) |
|---|---|
| A. muscaria extract | 120 |
| Permeation enhancing agent | 30.0 |
| Solubilizer | 20.0 |
| Stabilizer | To 100 |

The stabilizer, solubilizer, and permeation enhancing agent are heated and stirred until combined. A. muscaria extract is added (i.e., including additional active agents, if desired) after partially cooled but before setting and stirring is continued until dispersed. The mixture is then cooled until in its desired final form (e.g., for use in a reservoir delivery system) or admixed with an adhesive and then cooled (e.g., for use in a drug-in-adhesive patch).

### Example 19: Formulation of Topical Pain Transdermal Form

A topical formulation for transdermal administration may be prepared as follows. The emulsifiers Polawax and shea butter are provided to facilitate the mixing zinc oxide with water.

| Ingredient | Amount (%) |
|---|---|
| A. muscaria extract | 0.005% |
| | (5 micrograms/milliliter) |
| Water | 83.8% |
| Polawax | 5.0% |
| Shea butter | 3% |
| Ginkgo biloba | 0.1% |
| Zinc oxide | 8.0% |

In this example, the amount of A. muscaria extract provided in the cream is 5 micrograms/milliliter (0.005% by volume). Any value within the range from about 1.0 to about 10.0 micrograms per milliliter (about 0.001% to about 0.01% by volume) may be provided. Glycerol may be provided instead of or along with the Polawax and shea butter as emulsifier agents, with a total emulsifier agent content of about 8.0% in this example. Aloe gel may be used instead of or along with zinc oxide, with a total combined content of about 8.0% in this example.

### Example 20: A. Muscaria Formulation for Transdermal Pain

A transdermal formulation further includes an additional humectant, a fragrance, and a preservative. The additional humectant in this example is eucalyptus oil. The fragrance in this example is coconut cream, and the preservative in this example is citric oxide. The emulsifiers polawax and shea butter further facilitate the mixing of the eucalyptus oil and the coconut cream with the water. The cream formulation has the following formulation:

| Ingredient | Amount (%) |
|---|---|
| A. muscaria extract | 0.005% |
| | (5 micrograms/milliliter) |
| Water | 77.6% |
| Polawax | 5.0% |
| Shea butter | 3.0% |
| Ginkgo biloba | 0.1% |
| Zinc oxide | 8.0% |
| Coconut cream | 5.0% |
| Eucalyptus oil | 0.3% |
| Citric acid | 1.0 % |

### Example 21: Formulation of Transdermal Anti-Pruritus and/or Anti-Inflammatory Form

A transdermal anti-pruritis and/or anti-inflammatory formulation includes eucalyptus oil and Ginkgo biloba for their increased skin moisturization and soothing properties. Zinc oxide is provided for forming a protective barrier and for providing an anti-inflammatory effect.

| Ingredient | Amount (%) |
|---|---|
| A. muscaria extract | 0.005% |
| | (5 micrograms/milliliter) |
| Water | 83.6% |
| Polawax | 5.0% |
| Shea butter | 3% |
| Ginkgo biloba | 0.1% |
| Eucalyptus oil | 0.3% |
| Zinc oxide | 8.0% |

As above, the amount of A. muscaria extract provided may be in a range from about 1.0 to about 10.0 micrograms per milliliter, which is equivalent to from about 0.001% to about 0.01% by volume. Glycerol may be provided instead of or along with the Polawax and shea butter as emulsifier agents, with a total emulsifier agent content of about 8.0% in this example. Aloe gel may be used instead of or along with zinc oxide, with a total content of about 8.0% in this example. An ointment could have a petrolatum content of about 61.9% petrolatum and a water content of about 21.9%, for example.

### Example 22: Formulation of Transdermal Anti-Pruritus and/or Anti-Inflammatory Form

Another example of a transdermal formulation for alleviation of pruritus and/or inflammation, comprising moisturizers, anti-inflammatoires, fragrance, and a preservative. The anti-inflammatoires are kukui nut oil and sea buckthorn oil, which also moisturize the skin. The humectants in this example are eucalyptus oil and Ginkgo biloba. The fragrance in this example is coconut cream fragment oil, which also moisturizes the skin. The emulsifiers polawax and shea butter are provided to facilitate the mixing of the water soluble and oil soluble components. The preservative in this example is citric oxide. The cream formulation has the following composition by volume:

| Ingredient | Amount (%) |
|---|---|
| A. muscaria extract | 0.005% |
| | (5 micrograms/milliliter) |
| Water | 57.9% |
| Polawax | 5.0% |
| Shea butter | 3.0% |
| Ginkgo biloba | 0.1% |
| Eucalyptus oil | 0.3% |
| Zinc oxide | 8.0% |
| Kukui nut oil | 10.0% |
| Sea buckthorn oil | 10.0% |
| Coconut cream fragment oil | 5.0% |
| Citric acid | 1.0% |

### Example 23: Formulation of a Transdermal Rejuvenation/Moisturizing Form

A topical rejuvenation/moisturizing formulation is a transdermal formulation that includes aloe vera, cocoa seed butter, and glycerin for their moisturizing qualities. Aloe vera and hibiscus also have anti-inflammatory qualities. Glycerin acts as an emulsifier, along with polawax and shea butter. Citric acid is provided as a preservative.

| Ingredient | Amount (%) |
|---|---|
| A. muscaria extract | 0.005% |
| | (5 micrograms/milliliter) |
| Water | 79.0% |
| Polawax | 5.0% |
| Shea butter | 3.0% |
| Aloe vera | 5.0% |
| Cocoa seed butter | 1.0% |
| Glycerin | 5.0% |
| Hibiscus | 1.0% |
| Citric acid | 1.0% |

The amount of A. muscaria extract provided may be in a range from about 1.0 to about 10.0 micrograms per milliliter, which is equivalent to from about 0.001% to about 0.01% by volume. Glycerol may be provided instead of or along with the Polawax and shea butter as emulsifier agents to facilitate the mixing of the water soluble and oil soluble components, with a total emulsifier agent content of about 8.0% in this example. Aloe gel may be used instead of or along with zinc oxide, with a total content of about 8.0% in this example.

### Example 24: Formulation of a Transdermal Rejuvenation/Moisturizing Form

A transdermal rejuvenation/moisturizing formulation that further includes the anti-inflammatoires black cumin, Aleurites molusccanus Seed Oil, jojoba oil, rosa canina, and sativa seed extract. Lavender is also included, which is both anti-inflammatory and a fragrance.

| Ingredient | Amount (%) |
|---|---|
| A. muscaria extract | 0.005% |
| | (5 micrograms/milliliter) |
| Water | 78.4% |
| Polawax | 5.0% |
| Shea butter | 3.0% |
| Black cumin | 10% |
| Aloe vera | 5.0% |
| Cocoa seed butter | 1.0% |
| Glycerin | 5.0% |
| Jojoba oil | 5.0% |
| Aleurites molusccanus | 2.0% |
| seed oil | |
| Sativa seed extract | 1.0% |
| Rosa canina | 0.65% |
| Hibiscus | 1.0% |
| Parfum | 2.0% |
| Lavender | 2.0% |
| Radish root | 1.0% |
| Citric acid | 1.0% |

### Example 25: Formulation of a Transdermal Rejuvenation/Moisturizing Form

A transdermal rejuvenation/moisturizing formulation that further includes aloe vera, cocoa seed butter, and glycerin for their moisturizing qualities. Aloe vera and hibiscus also have anti-inflammatory qualities. Glycerin also acts as an emulsifier. Citric acid is provided as a preservative.

| Ingredient | Amount (%) |
|---|---|
| A. muscaria extract | 0.005% |
| | (5 micrograms/milliliter) |
| Water | 78.0% |
| Polawax | 5.0% |
| Shea butter | 3.0% |
| Aloe vera | 5.0% |
| Cocoa seed butter | 1.0% |
| Glycerin | 5.0% |
| Hibiscus | 1.0% |
| Citric acid | 1.0% |

As above, the amount of A. muscaria extract provided may be in a range from about 1.0 to about 10.0 μg per mL, which is equivalent to from about 0.001% to about 0.01% by volume. Glycerol may be provided instead of or along with Polawax and shea butter as emulsifier agents, with a total emulsifier agent content of about 8.0% in this example. Aloe gel may be used instead of or along with zinc oxide, with a total content of about 8.0% in this example.

### Example 26: Formulation of Transdermal Immune Enhancement Form

A transdermal immune enhancement formulation includes cat's claw, hibiscus, and nettle leaf for their immune enhancing characteristics. Black cumin is provided for its anti-inflammatory effect. Citric acid is provided as a preservative.

| Ingredient | Amount (%) |
|---|---|
| A. muscaria extract | 0.005% |
| | (5 micrograms/milliliter) |
| Water | 78.8% |
| Polawax | 5.0% |
| Shea butter | 3.0% |
| Black cumin | 10.0% |
| Cat's claw | 50 mg |
| Hibiscus | 1.0% |
| Nettle leaf | 1.0% |
| Citric acid | 1.0% |

As in the above formulations, the amount of A. muscaria extract provided in a range from about 1.0 to about 10.0 micrograms per milliliter, which is equivalent to from about 0.001% to about 0.01% by volume. The A. muscaria extract is about 0.005% (5 micrograms/milliliter). Glycerol may be provided instead of or along with the Polawax and shea butter as emulsifier agents, with a total emulsifier agent content of about 8.0% in this example.

### Example 27: Formulation of Transdermal Immune Enhancement Form

A transdermal immune enhancement formulation that further includes the anti-inflammatories moringa oil and spearmint oil. Aloe vera is included to provide a protective layer and for moisturization of the skin. Parfum is also included as a fragrance.

| Ingredient | Amount (%) |
|---|---|
| A. muscaria extract | 0.005% |
| | (5 micrograms/milliliter) |
| Water | 68.7% |
| Polawax | 5.0% |
| Shea butter | 3.0% |
| Black cumin | 10.0% |
| Cat's claw | 50 mg |
| Hibiscus | 1.0% |
| Nettle leaf | 1.0% |
| Moringa oil | 1.0% |
| Aloe vera | 5.0% |
| Ginkgo biloba | 0.1% |
| Spearmint oil | 2.0% |
| Parfum | 2.0% |
| Citric acid | 1.0% |

As above, the amount of A. muscaria extract provided may be in a range from about 1.0 to about 10.0 micrograms per milliliter, which is equivalent to from about 0.001% to about 0.01% by volume. Glycerol may be used instead of or along with the Polawax and shea butter as emulsifier agent(s), with a total emulsifier agent content of about 8.0% in this example.

The active ingredient(s) are added (i.e., including additional active agents, if desired) after partially cooled but before setting and stirring is continued until dispersed. The stabilizer, solubilizer, and/or permeation enhancing agent, if provided, are added and stirred until combined. The mixture is then cooled until in its desired final form (e.g., for use in a reservoir delivery system) or admixed with an adhesive and then cooled (e.g., for use in a drug-in-adhesive patch).

### Example 28: Formulation of Cut Matrix Sublingual or Buccal Tablets

Sublingual or buccal tablets are made as a single matrix and then cut to size:

| Ingredient | Amount (mg/tablet) |
|---|---|
| A. muscaria extract | 120 |
| Glycerol | 210.5 |
| Water | 143.0 |
| Sodium citrate | 4.5 |
| Polyvinyl alcohol | 26.5 |
| Polyvinylpyrrolidone | 15.5 |

The glycerol, water, sodium citrate, polyvinyl alcohol, and polyvinylpyrrolidone are admixed together by continuous stirring and maintaining the temperature at about 90° C. When the polymers have gone into solution, the solution is cooled to about 50-55° C. and the A. muscaria extract is slowly admixed. The homogenous mixture is poured into forms made of an inert material to produce a drug-containing diffusion matrix having a thickness of about 2-4 mm. This diffusion matrix is then cut to form individual tablets having the appropriate size.

### Example 29: Formulation of Individually Formed Sublingual or Buccal Lozenges

Sublingual or buccal lozenges are made from individual forms or molds:

| Ingredient | Amount (mg/each lozenge) |
|---|---|
| A. muscaria extract | 120 |
| Silica gel powder | 350 |
| Citric acid powder | 400 |
| Acacia powder | 600 |
| Flavor (optional) | 100 |
| Polyethylene glycol | 1,000 |

The inactive ingredients are admixed by continuous stirring and maintaining the temperature at about 90° C. When the PEG has melted and the other ingredients have gone into solution, the solution is cooled to about 50-55° C. and the A. muscaria extract is slowly admixed. The homogenous mixture is poured into separate molds and allowed to cool. Reference may also be made to U.S. Pat. No. 10,034,832 and the Examples therein, all of which is incorporated herein.

### Example 30: Formulation of Confectionery Products and Gummies

Confectionery products such as chewy “gummies” can be made as follows:

| Ingredient | Amount (mg/each gummy) |
|---|---|
| A. muscaria extract | 250 mg |
| Sugar | 675 |
| Corn syrup 42 DE | 1,000 |
| Fruit juice concentrate | 200 |
| (65-70% solids) | |
| Distilled or deionized water | 175 |
| Palm kernel oil | 150 |
| Gelatin (250 bloom) | 40 |
| Citric acid | 25 |
| Malic acid | 7.5 |
| Flavoring | 7 |
| Monoglyceride | 4 |
| Lecithin | 3 |
| Colorants | 2 |

The gelatin is dissolved in water and added to a mixer. Next, a syrup is made in a vacuum cooking system by continuously mixing and cooking sugar, water, and the corn syrup to a temperature of about 250° F.-275° F. The sugar, corn syrup and other liquids are cooked in a jacketed mixing kettle with an agitator to a temperature of about 125° F.-150° F. and cooked to a final temperature of about 250° F.-275° F. in a vacuum cooking system. This syrup is added to the dissolved gelatin in the mixer and is blended until the mixture thickens (for about 3 minutes). The mixture is then blended at high speed until it is aerated (for about 2 minutes). Next, the mixer speed is lowered and the remaining ingredients are added to form a paste, which is mixed until homogenous (for about 2 minutes). The homogenous mixture is pumped out of the mixer, and then formed into desired shapes by using a puller unit, a batch roller, rope sizers, and/or a die former (drop roller or ball former). Other such products, including candy coated confectionery products, are disclosed in U.S. Pub. No. 2005/0191406, which is incorporated by reference.

### Example 31: Vaporizer Formulation

50:50 base liquid vaporizer formulation comprising 50 mg/mL compound can be prepared as follows:

| Ingredient | Quantity (units) |
|---|---|
| A. muscaria extract | 500 mg |
| 50:50 base liquid (50% PG, 50% VG) | 10 mL |

The ingredients are mixed and prepared for use with any liquid vaporization device or appliance, such as e-liquid vaporizers, e-cigs, mods, vape pens, and the like, and can be formulated for any other oil, thin oil, “e-juice,” or e-liquid vaporizer, according to ordinary skill. Flavoring(s) optionally may be added if desired, and water and/or ethanol may be added to the base liquid as a diluent.

### Example 32: Alternative Vaporizer Formulation

50:50 base liquid vaporizer formulation comprising 10% A. muscaria extract can be prepared as follows:

| Ingredient | Percentage v/v |
|---|---|
| A. muscaria extract | 10% |
| 50:50 base liquid (50% PG, 50% VG) | 90% |

It will be appreciated that the above example may be produced in any desired volume, and the ingredients may be combined with tools known to those in the art. As an example, if the desired volume is 10 mL, 4.5 mL PG, 4.5 mL VG, and 1 mL A. muscaria extract will be combined. Alternatively, one may include in the base liquid any of PEG, ethanol, and water, depending on the specific desired embodiment.

### Example 33: Formulation of Intranasal Delivery Form

A nasal spray formulation for intranasal delivery may be prepared as follows:

| Ingredient | Quantity (units) |
|---|---|
| A. muscaria extract | 500 mg |
| DMSO | 50 μL |
| MCT | 5 mL |
| Saline (1% cremophor) | To 10 mL |

The solution at 10 mg/ml of active ingredients in 49.5% MCT, 49.5% saline, 0.5% DMSO, and 0.5% cremophor is prepared, as above (but with MCT in place of TEG), for use in nasal spray device. In other embodiments, a nasal formulation can be prepared as a dry powder for inhalation, e.g., by combining compound with lactose and mixing for use with a dry powder inhaling appliance, or as in U.S. Pub. No. US2015/0367091A1 and references cited therein.

### Example 34: Formulation of Liquid or Dry Powder Beverage Concentrate

A formulation suitable for use as a beverage concentrate powder is made as follows:

| Ingredient | Quantity (mg) |
|---|---|
| A. muscaria extract | 870.0 |
| Glycerin | 55.0 |
| Sweetener | 40.0 |
| Flavorings | 2.0 |
| Colorants | 1.0 |
| Salt | 0.5 |
| Stabilizing gum | 0.3 |
| Preservatives | 0.2 |

Formulations are prepared with ingredients admixed and blended until consistent, the above ingredients being merely exemplary. Optional additional components include further flavorings and colorants, further preservatives, acidulants, and emulsifying agents. The limitations on the additions of all of these optional components are variable dependent on their final effect on taste, mouthfeel, and viscosity, which they should not adversely affect in the final beverage product. In embodiments, the overall viscosity of the liquid beverage concentrate should remain sufficiently low to enable the use of conventional beverage pumps and dispensers.

In embodiments, a final beverage product will be created by mixing the concentrate with between about two and about 20 volumes of water, or more as will vary by personal preference. In alternative embodiments, a concentrate can be added to another liquid such as juice, tea, soda, a sports or electrolyte drink, a cocktail or alcoholic beverage, or the like.

Concentrates can also be prepared and added to prepackaged beverages such as teas, juices, water, sports drinks and electrolyte drinks, single-serving beverage pouches and “shots,” alcoholic beverages, and the like, and whether for a single or multiple serving (it also will be readily appreciated that in other embodiments, compound can be added directly to any beverage without additional preparation or formulation).

### Example 35: Formulation of Syrup

Syrup containing an A. muscaria extract can be made as follows:

| Ingredient | Quantity (units) |
|---|---|
| A. muscaria extract | 2 g |
| Filtered water | 750 mL |
| Fine granulated sugar | 750 mL |
| Vegetable glycerin | 30 mL |

The water and sugar are brought to the boil, and stirred consistently, until fully dissolved. The temperature is reduced to a simmer and the A. muscaria extract is added, then stirred until fully blended into the mixture. Vegetable glycerin is added. Mixture is simmered for 5 to 6 minutes, and stirred briefly and briskly at each minute. The hot syrup is passed through a No. 20 mesh U.S. sieve into a container. The resulting syrup is allowed to cool, and can be added to beverages or used in baking.

### Example 36: Formulation of Elixir

Elixir is made as follows:

| Ingredient | Quantity (units) |
|---|---|
| A. muscaria extract | 10 g |
| Coconut oil | 177 mL |
| Unrefined vegetable oil (e.g., hemp seed oil) | 532 mL |
| Lecithin liquid | 15 mL |
| Grain alcohol (190 proof/95% ethanol). | 355 mL |

The A. muscaria extract is placed in a boiling water bath. The coconut oil is melted and mixed with A. muscaria extract in a crucible. Lecithin liquid is added to the crucible to form a mixture. Unrefined vegetable oil is added to the mixture and mixing to form the cannabis elixir. The elixir prepared using the techniques described above may be used topically, orally as a tincture, or in pill form. To double the strength of the elixir, the quantity of the oils and lecithin may be reduced to half. The elixir should be brilliantly clear and can be strained or filtered, or further clarified through the addition of purified talc or siliceous earth. Sorbitol, glycerin, sucrose or artificial sweeteners may also be used to sweeten the mixture. An adjuvant solvent (propylene glycol) may be added to reach a desired volume.

### Example 37: Formulation of Oral Thin Film

An oral thin film is made as follows:

| Ingredient | Quantity (100 mg thin film) | |
|---|---|---|
| A. muscaria extract | up to about 65 wt. % | |
| Purified water | 2-24 | wt. % |
| Pectin | 4-50 | wt. % |
| Vegetable oil | 0-22 | wt. % |
| Glycerin | 3-22 | wt. % |
| Microcrystalline cellulose | 0-20 | wt. % |
| Sodium benzoate | 0-0.1 | wt. % |
| Flavoring agent | 0-10 | wt. % |
| Coloring agent | 0-1.0 | wt. % |

The vegetable oil, glycerin and purified water are blended to provide a uniform first mixture. The mixing will typically be carried out at an elevated temperature (e.g., about 130-140° F. or 54.4-60° C.) The mixing can be carried out in any suitable manner, employing equipment known to those of skill, e.g., blending in a blender. The A. muscaria extract is then added to the mixture at room temperature (e.g., about 70° F.) for a suitable period of time (e.g., up to about 5 minutes) sufficient to provide the thickened second mixture. Pectin, microcrystalline cellulose, flavoring agents, and sweetening agents are then added, thickening the second mixture, creating a slurry. The slurry is cooled, sheared, mixed, cast, and condensed to provide an oral thin film, which can then be cut. Alternatively, the slurry is hot extruded, cast, and condensed to provide an oral thin film, which can then be cut. Alternatively, the vegetable oil can be replaced with other lipids such as deodorized cocoa butter oil or fruit seed oil. Similarly, glycerin can be replaced with ethoxylated monoglycerides or ethoxylated diglycerides. The thin film described herein can optionally further include a mucoadhesive agent. The mucoadhesive agent, when placed in the oral cavity in contact with the mucosa therein, adheres to the mucosa. Alternatively, up to about 10 wt. % of an A. muscaria compound may be used in place of A. muscaria extract, following the same procedure outlined above.

### Example 38: Formulation of a Troche

A single troche is made as follows:

| Ingredient | Quantity (mg per troche) |
|---|---|
| A. muscaria extract | 880 |
| Polyethylene glycol 1450 | 1000 |
| Stevia | 100 |
| Acacia gum | 600 |
| Citric acid | 400 |

A base is prepared by melting polyethylene glycol 1450, with stevia, acacia gum, and citric acid at a temperature between 58° C. and 64° C. at normal atmospheric pressure. An A. muscaria extract is then added to form a uniform mixture, with the temperature maintained between approximately 58° C. and 63° C.

The resultant solution is then poured into a lozenge or troche mold device, using a micropipette. The mixture is allowed to cool at room temperature.

### Example 39: Formulation of a Caplet

A single caplet is made as follows:

| Ingredient | Quantity (mg/caplet) |
|---|---|
| A. muscaria extract | 880.0 |
| Starch | 45.0 |
| Microcrystalline cellulose | 30.0 |
| Magnesium stearate | 0.5 |
| Silicon dioxide | 10.0 |
| Acacia gum | 2.0 |

A. muscaria extract, starch, magnesium stearate, silicon dioxide and acacia gum are blended and passed through a No. 20 mesh U.S. sieve. High pressure is applied to the powder bed, compressing the powder into a coherent compact. Additional excipients such as sucrose, talc, or titanium dioxide, may also be used to coat the caplet.

### Example 40: Formulation of a Functional Food

In embodiments, the disclosed A. muscaria compositions are used as functional foods. In embodiments, the disclosed A. muscaria compositions are used in the manufacture of a functional food. In embodiments, the disclosed A. muscaria compositions are administered to a subject as a functional food. In embodiments, a functional food comprises a disclosed A. muscaria extract. In embodiments, a functional food comprises a disclosed A. muscaria compound, an analog thereof, or any combination thereof. In embodiments, a functional food provides the benefits described herein to a subject who ingests said functional food.

In embodiments, the functional food comprising the disclosed A. muscaria compositions is a dairy product. Exemplary dairy products include milk, yogurt, and cream cheese. In some embodiments the functional food comprising the disclosed A. muscaria compositions is a grain product. Exemplary grain products include bread, noodles, and cereal.

One exemplary functional food containing A. muscaria extract is as follows:

| Ingredient | Quantity (per powdered serving) |
|---|---|
| A. muscaria extract | 880 mg |
| Carbohydrate source (e.g., FOS) | 40-80% |
| Fat (e.g., coconut oil) | 20-60% |
| Protein (e.g., casein) | 1-10% |
| Emulsifier (e.g., mono or diglycerides) | 1-2% |
| Stabilizer (e.g., sodium polyphosphate) | 2-3% |
| Free flowing agent (e.g., silicon dioxide) | 0.5-1% |
| Water | Calculated & added to achieve |
| | solid total of 60-72% |

All ingredients, except the A. muscaria extract, are added and blended using a homo-mixer to provide an emulsion with a solid total of 60-72%. FOS refers to fructo-oligosaccharides. Remixing is performed to produce a more uniform emulsion mixture by passing the mixture through homogenized pressure of between 150-250 bars. Sterilization is performed to kill microbes. The mixture is pasteurized at a temperature of 75-90° C. for at least 30 seconds. The mixture is then pumped. The mixture is then dried to evaporate the water content to obtain the final product in the form of a powder. The drying process is carried out using a spray dryer unit with inlet air temperature of 150-200° C. and the discharge air temperature is 88-95° C. During the drying process the moisture content of the product is maintained at 1.5% to 5.0%. Finally, the powder is sieved to create a product with a uniform grain. During the sieving process, a A. muscaria extract is added. The functional food can be consumed directly by dissolving 10 grams of powder into 100 ml of hot water with a temperature of 85±5° C. In addition to direct consumption, functional foods can be added to drinks or processed food products. The product is prepared so that a single serving size comprises 880 mg of the A. muscaria extract.

### Example 41: Formulation of an Effervescent Powder (Wet and Dry)

An effervescent powder is made as follows:

| Ingredient | Quantity (w/v %) |
|---|---|
| A. muscaria extract | 20.0 |
| Citric acid | 12.0 |
| Tartaric acid | 24.0 |
| Sodium hydrogen carbonate | 41.0 |
| PVP30 | 3.0 |

In order to prepare the effervescent mixture, the alkaline component and acid component are prepared in a stoichiometric ratio, in which the alkaline component is present in a stoichiometric excess to that of the acid component. An A. muscaria extract (and any additional active agent) is incorporated therein and dry blended. Optional flavorings, sweeteners, preservatives, stabilizers and antioxidants, if desired, are incorporated at this point. Alternatively, if a wet granulation is performed, a solution containing a selected binder and solvent is prepared and added to the mixture. The preparation is mixed until suitable granulation is achieved. Flavorants, sweeteners, preservatives and antioxidants, if added, are incorporated in the binder/solvent solution. The granules are then dried, milled, and screened to the desired size. The quantity of A. muscaria extract included in the effervescent formulation may range from about 10% to about 65% by weight. Suitable bicarbonates appropriate for the formulation of the invention include sodium bicarbonate, calcium bicarbonate, potassium bicarbonate, barium bicarbonate, strontium bicarbonate, and magnesium bicarbonate. Other acids which may be used include citric, ascorbic, malic, succinic, phosphoric, and monopotassium phosphate. Organic acids such as citric, tartaric and malic acids are particularly suitable.

### Example 42: Formulation of Nutritional Supplement

A. muscaria extract dietary supplements are made as follows:

| Ingredient | Quantity (mg/capsule) |
|---|---|
| A. muscaria extract | 440 mg |
| Cellulose | 119.0 mg |
| Magnesium stearate | 1.0 mg |

A. muscaria extract and magnesium stearate are blended, passed through a No. 20 mesh U.S. sieve, and inserted into a hard cellulose capsule. Additional active ingredients such as herbs, vitamins, enzymes, amino acids, prebiotics, probiotics, or minerals may also be added at the same time as the A. muscaria extract and magnesium stearate. Two capsules may be administered to a subject or self-administered by a subject for a total of 880 mg of extract.

### Example 43: Formulation of a Nutritional Softgel Supplements

A. muscaria extract nutritional softgel supplements are made as follows:

| Ingredient | Quantity (units) | |
|---|---|---|
| A. muscaria extract | 500 | mg |
| Potato starch | 10 | wt. % |
| Gelatin (170-180 bloom) | 34 | wt. % |
| Purified water | 26 | wt. % |
| Glycerin (99.5%) | 28 | wt. % |
| Oil | 1.5 | wt. % |
| Aspartame | 0.25 | wt. % |
| Flavor | 0.25 | wt. % |

A mixture of gelatin, glycerin, oil, aspartame and flavor (up to a weight of 200 kg) are placed in a cooking tank with 800 L of capacity. The cooking tank is heated to 80-85° C. and the temperature is maintained for a period of 2-3 hours. The temperature of the cooking tank is then reduced to 55° C. until all air bubbles are completely removed. Softgel capsules of 20-oval size are then produced using conventional soft capsule machinery. The softgel capsules are filled with an A. muscaria extract.

The softgel capsules may further contain additional active agents such as proteins or amino acids, carbohydrates, lipids, vitamins, minerals and cofactors, natural or artificial flavors, dyes or other coloring additives, and preservatives. Flavors incorporated in the softgel capsules may include flavor concentrates known to those of skill, for example, flavor concentrates that replicate different food and drink flavors. Suitable oils for incorporation into a softgel capsule in an embodiment of the invention include an edible oil, extracts, and oil concentrations or combinations, blends, or mixtures thereof. Additional excipients may also be added including disintegrating agents such as cellulose and fillers such as starch.

### Example 44: Formulation of a Nutraceutical Powder

A nutraceutical powder comprising an A. muscaria extract is made as follows:

| Ingredient | Quantity (per 77 gram scoop) |
|---|---|
| A. muscaria extract | 10% wt |
| Carbohydrate (such as sweet potato flour) | 30% wt |
| Fat (such as flax seed powder) | 30% wt |
| Protein (such as egg white protein powder) | 30% wt |

A nutraceutical formulation is provided by simultaneously combining A. muscaria extract with carbohydrates, fats, and proteins in powdered format. A. muscaria extract can be used alone or in combination with other active agents, such as fatty acids, adaptogens, minerals, vitamins, amino acids, enzymes, probiotics or prebiotics. Additional excipients such as minerals, flavoring agents, non-protein mass, or free-flowing agents such as magnesium stearate may also be added. The listed carbohydrates, fats, and proteins, may be substituted with appropriate alternatives. The nutraceutical powder may be mixed with a liquid prior to consumption. In one embodiment, two scoops of 77 grams each of the nutraceutical formulation powder are mixed with 12-16 oz of cold water or any suitable liquid, including a milk such as cow's milk, almond milk, coconut milk, hemp milk, soy milk, rice milk, or cashew milk; yogurt or kefir; or a smoothie or shake.

### Example 45: Formulation of Infused Chocolate

A. muscaria extract infused chocolate is made as follows:

| Ingredient | Quantity (per 50 gram chocolate bar) |
|---|---|
| A. muscaria extract | 250 mg |
| Coconut oil | 3000 mg |
| Chocolate | 46450 mg |
| Lecithin | 200 mg |
| Xanthan gum | 100 mg |

The coconut oil is heated to between 12° and 220° F. A. muscaria extract is added, followed by the emulsifiers. The emulsifiers can be used individually or in combination. The heated mixture is blended in a high-speed blender for two minutes. The mixture is allowed to cool to room temperature. The chocolate is then melted and added, before being allowed to temper. The resultant mixture is then deposited in a mold then cooled to 55° F.

The foregoing description, for purposes of explanation, uses specific nomenclature to provide a thorough understanding of the invention. However, it will be apparent to one skilled in the art that specific details are not required in order to practice the invention. Thus, the foregoing description of specific embodiments of the invention is presented for purposes of illustration and description. It is not intended to be exhaustive or to limit the invention to the precise forms disclosed; obviously, many modifications and variations are possible in view of the above teachings. The embodiments were chosen and described in order to best explain the principles of the invention and its practical applications, through the elucidation of specific examples, and to thereby enable others skilled in the art to best utilize the invention and various embodiments with various modifications as are suited to the particular use contemplated, when such uses are beyond the specific examples disclosed. Accordingly, the scope of the invention shall be defined solely by the following claims and their equivalents.

The invention claimed is:

1. A method for producing a wellness-promoting Amanita muscaria extract from mushroom biomass, comprising:
i. selecting mushroom biomass for extraction;
ii. performing an aqueous extraction of the biomass in the presence of heat to produce the extract;
iii. filtering the extract;
iv. reducing the pH of the extract;
V. heating the extract to reflux; and
vi. collecting the wellness-promoting Amanita muscaria extract.

2. The method of claim 1, wherein the biomass is dehydrated prior to performing the aqueous extraction.

3. The method of claim 2, wherein the biomass is dehydrated to a moisture content of about 1% to about 10%, about 2% to 7%, about 4% to 7%, or about 2%, 3%, or 4% prior to performing the aqueous extraction.

4. The method of claim 1, wherein the biomass is ground into a powder prior to performing the aqueous extraction.

5. The method of claim 1, wherein selecting the biomass for extraction comprises subjecting the biomass to a quality control analysis.

6. The method of claim 5, wherein the analysis comprises chemical analysis.

7. The method of claim 6, wherein the chemical analysis comprises a chromatography technique.

8. The method of claim 6, wherein the chemical analysis comprises any of high-performance liquid chromatography-mass spectrometry, liquid chromatography-mass spectrometry, and gas chromatography-mass spectrometry.

9. The method of claim 6, wherein the chemical analysis comprises determining the concentration of stizolobic acid in the biomass.

10. The method of claim 9, wherein the biomass is selected for extraction if the concentration of stizolobic acid in the biomass is less than or equal to 100 μg/g.

11. The method of claim 9, wherein the biomass is selected for extraction if the concentration of stizolobic acid in the biomass is less than or equal to 50 μg/g.

12. The method of claim 9, wherein the chemical analysis comprises determining the concentration of stizolobinic acid in the biomass.

13. The method of claim 12, wherein the biomass is selected for extraction if the concentration of stizolobinic acid in the biomass is less than or equal to 100 μg/g.

14. The method of claim 12, wherein the biomass is selected for extraction if the concentration of stizolobinic acid in the biomass is less than or equal to 50 μg/g.

15. The method of claim 6, wherein the chemical analysis comprises determining the concentration of any of cadmium, arsenic, lead, and mercury in the biomass.

16. The method of claim 6, wherein the chemical analysis comprises inductively coupled plasma mass spectrometry.

17. The method of claim 15, wherein the biomass is selected for extraction if the concentration of cadmium is less than or equal to about 2.0 ppm, 1.5 ppm, 1.0 ppm, or 0.5 ppm.

18. The method of claim 17, wherein the biomass is selected for extraction if the concentration of cadmium is less than or equal to about 0.5 ppm.

19. The method of claim 15, wherein the biomass is selected for extraction if the concentration of arsenic is less than or equal to about 5.0 ppm, 4.0 ppm, 3.0 ppm, 2.0 ppm, 1.5 ppm, 1.0 ppm, or 0.5 ppm.

20. The method of claim 19, wherein the biomass is selected for extraction if the concentration of arsenic is less than or equal to about 1.5 ppm.

21. The method of claim 15, wherein the biomass is selected for extraction if the concentration of lead is less than or equal to about 2.0 ppm, 1.5 ppm, 1.0 ppm, or 0.5 ppm.

22. The method of claim 21, wherein the biomass is selected for extraction if the concentration of lead is less than or equal to about 0.5 ppm.

23. The method of claim 15, wherein the biomass is selected for extraction if the concentration of mercury is less than or equal to about 5.0 ppm, 4.0 ppm, 3.0 ppm, 2.0 ppm, 1.5 ppm, 1.0 ppm, or 0.5 ppm.

24. The method of claim 23, wherein the biomass is selected for extraction if the concentration of mercury is less than or equal to about 1.5 ppm.

25. The method of claim 6, wherein the chemical analysis comprises testing for the presence of Amanita pantherina in the biomass.

26. The method of claim 25, wherein the biomass is selected for extraction if the biomass comprises less than about 10%, 5%, 4%, 3%, 2%, or 1% (w/w) of Amanita pantherina.

27. The method of claim 25, wherein the biomass is selected for extraction if the biomass is substantially free of Amanita pantherina.

28. The method of claim 6, wherein the chemical analysis comprises determining the concentration of mycotoxins in the biomass.

29. The method of claim 28, wherein the biomass is selected for extraction if the biomass comprises less than about 20 parts per billion (ppb) of a mycotoxin.

30. The method of claim 6, wherein the chemical analysis comprises determining the concentration of pesticides in the biomass.

31. The method of claim 30, wherein the biomass is selected for extraction if the biomass is substantially free of pesticides.

32. The method of claim 5, wherein the analysis comprises an organoleptic analysis.

33. The method of claim 32, wherein the organoleptic analysis comprises evaluating any of the visual appearance, smell, taste, and texture of the biomass.

34. The method of claim 5, wherein the analysis comprises a microbiological analysis.

35. The method of claim 34, wherein the microbiological analysis comprises any of testing the biomass for the presence of Salmonella spp., testing for the presence of Shiga-toxin producing Escherichia coli (STEC) bacteria, determining the total coliform count, and determining the total aerobic plate count, determining the total yeast and mold count in the biomass.

36. The method of claim 1, wherein the aqueous extraction is performed in water heated to between about 70° C. and 100° C.

37. The method of claim 36, wherein the aqueous extraction is performed in water heated to about 80° C.

38. The method of claim 1, wherein the aqueous extraction is performed for between about 15 minutes and about 60 minutes.

39. The method of claim 1, wherein filtering the extract comprises passing the extract through a membrane filter.

40. The method of claim 39, wherein the membrane filter has a pore size of about 200 microns.

41. The method of claim 39, wherein the membrane filter is a sock filter.

42. The method of claim 41, wherein filtering the extract comprises passing the extract through two 200-micron sock filters in a parallel configuration.

43. The method of claim 1, wherein the pH of the extract is reduced to between about 2.0 and 3.0.

44. The method of claim 43, wherein the pH of the extract is reduced to between about 2.4 and 2.6.

45. The method of claim 1, wherein the pH of the extract is reduced using a mineral acid.

46. The method of claim 45, wherein the mineral acid is hydrochloric acid.

47. The method of claim 1, wherein the extract is refluxed at a temperature of between about 95° C. and 100° C.

48. The method of claim 1, wherein the extract is refluxed for between about 4.25 and 4.75 hours.

49. The method of claim 48, wherein the extract is refluxed for about 4.5 hours.

50. The method of claim 1, wherein refluxing the extract reduces the concentration of ibotenic acid and increases the concentration of muscimol in the extract.

51. The method of claim 1, further comprising increasing the pH of the extract.

52. The method of claim 51, wherein the pH of the extract is increased to between about 3.5 and 4.5.

53. The method of claim 52, wherein the pH of the extract is increased to about 4.

54. The method of claim 51, wherein the pH of the extract is increased by the addition of a hydroxide base.

55. The method of claim 1, wherein the pH of the extract is increased by the addition of sodium hydroxide pellets.

56. The method of claim 1, wherein the weight ratio of muscimol to ibotenic acid in the refluxed extract is between about 10:1 and 200:1.

57. The method of claim 1, wherein the weight ratio of muscimol to ibotenic acid in the refluxed extract is between about 10:1 and 100:1.

58. The method of claim 1, wherein the weight ratio of muscimol to ibotenic acid in the refluxed extract is between about 10:1 and 50:1.

59. The method of claim 1, wherein the weight ratio of muscimol to ibotenic acid in the refluxed extract is between about 10:1 and 25:1.

60. The method of claim 1, wherein the weight ratio of muscimol to ibotenic acid in the refluxed extract is between about 10:1 and 15:1.

61. The method of claim 1, wherein the refluxed extract comprises between about 100 and 130 μg/g of muscimol, between about 7 and 10 μg/g of muscarine, and between about 4 and 12 μg/g of ibotenic acid.

62. An extract of Amanita muscaria mushroom biomass obtained by the method of any one of claims 1-61.

63. The extract of claim 62, comprising muscimol and ibotenic acid in a weight ratio of between about 10:1 and 200:1.

64. The extract of claim 63, comprising muscimol and ibotenic acid in a weight ratio of between about 10:1 and 100:1.

65. The extract of claim 64, comprising muscimol and ibotenic acid in a weight ratio of between about 10:1 and 50:1.

66. The extract of claim 65, comprising muscimol and ibotenic acid in a weight ratio of between about 10:1 and 25:1.

67. The extract of claim 66, comprising muscimol and ibotenic acid in a weight ratio of between about 10:1 and 15:1.

68. The extract of claim 62, comprising between about 100 and 130 μg/g of muscimol, between about 7 and 10 μg/g of muscarine, and between about 4 and 12 μg/g of ibotenic acid.

69. The extract of any claim 62, further comprising a preservative.

70. The extract of claim 69, wherein the preservative is sodium benzoate, potassium sorbate, or a combination thereof.

71. The extract of claim 62, further comprising a flavoring agent.

72. The extract of claim 71, wherein the flavoring agent is a corn flavor.

73. A beverage formulation comprising the extract of claim 62.

74. The beverage formulation of claim 73, further comprising a flavoring agent.

75. The beverage formulation of claim 74, wherein the flavoring agent is a flavor of plum, prune, date, currant, fig, grape, raisin, cranberry, pineapple, peach, nectarine, banana, apple, pear, guava, apricot, Saskatoon berry, blueberry, plains berry, prairie berry, mulberry, elderberry, Barbados cherry (acerola cherry), choke cherry, chocolate, vanilla, caramel, coconut, olive, raspberry, strawberry, huckleberry, loganberry, dewberry, boysenberry, kiwi, cherry, blackberry, honey dew, green tea, cucumber, quince, buckthorn, passion fruit, sloe, rowan, gooseberry, pomegranate, persimmon, mango, rhubarb, papaya, litchi, lemon, orange, lime, tangerine, mandarin and grapefruit juices, or any combination thereof.

76. The beverage formulation of claim 75, wherein the flavoring agent is a mango flavor.

77. The beverage formulation of claim 73, further comprising magnesium.

78. The beverage formulation of claim 73, further comprising L-theanine.

79. A dietary supplement comprising the extract of claim 62.

80. An edible formulation comprising the extract of claim 62.

81. The edible formulation of claim 80, wherein the edible formulation is a gummy.

82. The edible formulation of claim 90, wherein the edible formulation is a liquid tincture.

83. A topical formulation comprising the extract of any claim 62.

84. The topical formulation of claim 83, wherein the topical formulation is a skin cream.

85. A vaporizer formulation comprising the extract of claim 62.
